(19) United States
(12) Patent Application Publication
(10) Pub. No.: US 2024/0182969 A1
(43) Pub. Date: Jun. 6, 2024

(54) METHOD FOR DETECTING ALLELES ASSOCIATED WITH KERATOCONUS

(71) Applicant: Avellino Lab USA, Inc., Menlo Park, CA (US)

(72) Inventor: Larry DEDIONISIO, Guerneville, CA (US)

(21) Appl. No.: 18/420,290

(22) Filed: Jan. 23, 2024

Related U.S. Application Data
(63) Continuation of application No. PCT/US2022/038187, filed on Jul. 25, 2022.
(60) Provisional application No. 63/225,426, filed on Jul. 23, 2021.
(60) Provisional application No. 63/231,235, filed on Aug. 9, 2021.

(51) Int. Cl. C12Q 1/6883; C12Q 1/6858

(52) U.S. Cl. CPC C12Q 1/6883; C12Q 1/6858; C12Q 2600/156

(57) ABSTRACT

Methods for treating keratoconus in a subject based on diagnosing or prognosing of keratoconus in the subject or predicting risk of keratoconus in the subject in accordance with detecting genetic variants in a sample from the subject are described.

### RELATED APPLICATIONS

This application is a continuation application of the international application PCT/US2022/038187, filed on Jul. 25, 2022, which claims the benefit of, and priority to, U.S. Provisional Application Ser. No. 63/225,426, filed on Jul. 23, 2021 and U.S. Provisional Application Ser. No. 63/231,235, filed on Aug. 9, 2021, all of which are incorporated by reference herein in their entireties.

### FIELD OF THE APPLICATION

This application generally relates to methods for the detection of an alleles associated with keratoconus diagnosis and prognosis. In particular, this application relates to methods for the treatment of keratoconus based on the detection of an alleles associated with keratoconus diagnosis and prognosis.

### BACKGROUND

Keratoconus (KC) is the most common corneal ectatic disorder with approximately 6-23.5% of subjects carrying a positive family history (Wheeler, J., Hauser, M. A., Afshari, N. A., Allingham, R. R., Liu, Y., Reproductive Sys Sexual Disord 2012; S:6). The reported prevalence of KC ranges from 8.8 to 54.4 per 100,000. This variation in prevalence is partly due to the different criteria used to diagnose the disease. (Wheeler, J., Hauser, M. A., Afshari, N. A., Allingham, R. R., Liu, Y., Reproductive Sys Sexual Disord 2012; S:6; and Nowak, D., Gajecka, M., Middle East Afr J Ophthalmol 2011; 18(1): 2-6). Many studies exist within the literature that attempt to define the genetic causes of KC. These studies have uncovered numerous possible genetic variants or SNPs that are believed to contribute to the etiology of the disease depending on the experimental parameters.

KC is a common corneal disorder where the central or paracentral cornea undergoes progressive thinning and steepening causing irregular astigmatism. The hereditary pattern is neither prominent nor predictable, but positive family histories have been reported. The incidence of KC is often reported to be 1 in 2000 people. KC can show the following pathologic findings, including, fragmentation of Bowman's layer, thinning of stroma and overlying epithelium, folds or breaks in Descemet's membrane, and variable amounts of diffuse corneal scarring.

Histopathology studies demonstrate breaks in or complete absence of Bowman's layer, collagen disorganization, scarring and thinning. The etiology of these changes is not known, though some suspect changes in enzymes that lead to breakdown of collagen in the cornea. While a genetic predisposition to KC is suggested, a specific gene has not been identified. The majority of KC cases are bilateral, but often asymmetric. The less affected eye may show a high amount of astigmatism or mild steepening. Onset is typically in early adolescence and progresses into the mid-20's and 30's. However, cases may begin much earlier or later in life. There is variable progression for each individual. There is often a history of frequent changes in eyeglasses which do not adequately correct vision. Another common progression is from soft contact lenses, to toric or astigmatism correcting contact lens, to rigid gas permeable contact lens.

No preventive strategy has been proven effective to date. Some feel that eye rubbing or pressure (e.g., sleeping with the hand against the eye) can cause and/or lead to progression of KC, so subjects should be informed not to rub the eyes. In some subjects, avoidance of allergens may help decrease eye irritation and therefore decrease eye rubbing.

At present, diagnosis can be made by slit-lamp examination and observation of central or inferior corneal thinning. Computerized videokeratography is also useful in detecting early KC and allows following its progression. Ultrasound pachymetry can also be used to measure the thinnest zone on the cornea. New algorithms using computerized videokeratography have been devised which now allow the detection of forme fruste, subclinical or suspected keratoconus. These devices may allow better screening of subjects for prospective refractive surgery, however there remains a need in the art for better prognostic and diagnostic methods.

The present disclosure meets this need and by providing methods for prognosis and diagnosis of KC by detection of mutated alleles associated with keratoconus.

### SUMMARY

The present disclosure provides improved methods for the detection of one or more alleles associated with KC.

In one aspect, the disclosure provides methods for detecting variants related to KC in a subject, the method comprising detecting ten or more genetic variants (e.g., single nucleotide polymorphisms (SNPs) and indels) in a sample from a subject, wherein the presence of ten or more genetic variants is indicative of KC in the subject.

In another aspect, the disclosure provides methods for detecting variants causing KC in a subject, the method comprising detecting ten or more genetic variants (e.g., single nucleotide polymorphisms (SNPs) and indels) in a sample from a subject, wherein the presence of ten or more genetic variants is indicative of KC in the subject.

In another aspect, the disclosure provides methods for diagnosing or prognosing KC in a subject, the method comprising detecting ten or more genetic variants (e.g., single nucleotide polymorphisms (SNPs) and indels) in a sample from a subject, wherein the presence of ten or more genetic variants is indicative of a diagnosis or prognosis of KC in the subject.

In another aspect, the disclosure provides methods for predicting risk of developing KC in a subject, the method comprising detecting ten or more genetic variants in a sample from a subject, wherein the presence of five or more of genetic variants is indicative of risk for the development of KC in the subject.

In another aspect, the disclosure provides methods for developing a treatment regimen for the treatment of KC in a subject, the method comprising detecting ten or more genetic variants in a sample from a subject, wherein the presence of five or more genetic variants is indicative of the need for a KC treatment regimen in the subject.

In some embodiments, the five or more genetic variants are of genes selected from the group consisting of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469. In some embodiments, the genetic variants are selected from at least 10, 15, 20, 25, 30, 35, 40, 41, 42, 43, 44, 45, 46, 47, 48, 49, 50, 51, 52, 53, 54, 55 or 56 genes selected from the group consisting of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469.

In some embodiments, the detecting described herein comprises detecting at least one genetic variant from each of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469.

In some embodiments, said variant detection is by a sequencing method.

In some embodiments, the genetic variants are selected from the group consisting of genetic variants listed in FIG. 1.

In some embodiments, the subject is Afro-American. In some embodiments, the subject is Caucasian. In some embodiments, the subject is Hispanic. In some embodiments, the subject is East Asian or Korean.

In some embodiments, the ten or more genetic variants are selected from the group consisting of any combination of the mutations (e.g., genetic variants) described herein (e.g., FIG. 1).

In some embodiments, the methods described herein further comprises amplifying a nucleotide molecule from the sample from the subject.

In some embodiments, the detecting described herein comprises detecting the genetic variants in a nucleotide molecule from the sample from the subject or its amplicons.

In some embodiments, the disclosure provides methods for treating keratoconus in a subject, the method comprising diagnosing or prognosing KC and treating KC in the subject. In further embodiments, the treatment may comprise wearing eye glasses or contact lenses, and/or performing collagen cross-linking or corneal transplant.

In one aspect, the disclosure provides methods for detecting variants related to KC in a subject, the method comprising detecting five or more genetic variants (e.g., single nucleotide polymorphisms (SNPs) and indels) in a sample from a subject, wherein the presence of five or more genetic variants is indicative of KC in the subject.

In another aspect, the disclosure provides methods for detecting variants causing KC in a subject, the method comprising detecting five or more genetic variants (e.g., single nucleotide polymorphisms (SNPs) and indels) in a sample from a subject, wherein the presence of five or more genetic variants is indicative of KC in the subject.

In another aspect, the disclosure provides methods for diagnosing or prognosing KC in a subject, the method comprising detecting five or more genetic variants (e.g., single nucleotide polymorphisms (SNPs) and indels) in a sample from a subject, wherein the presence of five or more genetic variants is indicative of a diagnosis or prognosis of KC in the subject.

In another aspect, the disclosure provides methods for predicting risk of developing KC in a subject, the method comprising detecting five or more genetic variants in a sample from a subject, wherein the presence of five or more of genetic variants is indicative of risk for the development of KC in the subject.

In another aspect, the disclosure provides methods for developing a treatment regimen for the treatment of KC in a subject, the method comprising detecting five or more genetic variants in a sample from a subject, wherein the presence of five or more genetic variants is indicative of the need for a KC treatment regimen in the subject.

In some embodiments, the five or more genetic variants are of genes selected from the group consisting of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469.

In some embodiments, the five or more genetic variants are of genes including at least one gene selected the group consisting of ABCA4, ABCB5, ABCC6, ADAMTS18, ADGRV1, ANGPTL7, BEST1, COL2A1, COL4A1, COL4A2, COL4A3, COL4A4, COL5A2, COL6A1, COL12A1, DIAPH1, DOCK9, FYN, HGF, ILIA, ILIRN, IL6, IL10, ITGB1, KRT13, KRT15, KRT16, KRT23, KRT24, LOX, LRRN1, LTBP2, MAP2K1, MAP3K19, MTOR, MYLK, NLRP1, PAX6, PIK3CG, PIK3R1, PTK2, PXDN, PXN, RAF1, RHOA, SFTPD, SHC1, SIX5, TLN1, WNT9A, and WNT9B.

In some embodiments, the genetic variants are selected from at least 10, 15, 20, 25, 30, 35, 40, 41, 42, 43, 44, 45, 46, 47, 48, 49, 50, 51, 52, 53, 54, 55 or 56 genes selected from the group consisting of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB 1, and ZNF469.

In some embodiments, the genetic variants are selected from at least 10, 15, 20, 25, 30, 35, 40, 41, 42, 43, 44, 45, 46, 47, 48, 49, 50, 51, 52, 53, 54, 55 or 56 genes selected from the group consisting of ABCA4, ABCB5, ABCC6, ADAMTS18, ADGRV1, ANGPTL7, BEST1, COL2A1, COL4A1, COL4A2, COL4A3, COL4A4, COL5A2, COL6A1, COL12A1, DIAPH1, DOCK9, FYN, HGF, ILIA, ILIRN, IL6, IL10, ITGB1, KRT13, KRT15, KRT16, KRT23, KRT24, LOX, LRRN1, LTBP2, MAP2K1, MAP3K19, MTOR, MYLK, NLRP1, PAX6, PIK3CG, PIK3R1, PTK2, PXDN, PXN, RAF1, RHOA, SFTPD, SHC1, SIX5, TLN1, WNT9A, and WNT9B.

### BRIEF DESCRIPTION OF THE DRAWINGS

FIG. 1 depicts exemplary generic variants.

FIGS. 2-26 depict an example and related experimental data verifying variants including those disclosed in FIG. 1.

### DETAILED DESCRIPTION

The detection of disease-related variants is an increasingly important tool for the diagnosis and prognosis of various medical conditions. With regard to KC, the present disclosure provides methods for detection of mutant alleles and use of this information in or to diagnose a subject with KC as well as to predict the risk of an individual in developing KC.

The term “invention” or “present invention” as used herein is not meant to be limiting to any one specific embodiment of the invention but applies generally to any and all embodiments of the invention as described in the claims and specification.

As used herein, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure. It should be understood that the use of “and/or” is defined inclusively such that the term “a, b and/or c” should be read to include the sets of “a,” “b,” “c,” “a and b,” “b and c,” “c and a,” and “a, b and c.”

As used herein, the term “about” means modifying, for example, lengths of nucleotide sequences, degrees of errors, dimensions, the quantity of an ingredient in a composition, concentrations, volumes, process temperature, process time, yields, flow rates, pressures, and like values, and ranges thereof, refers to variation in the numerical quantity that may occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of, for example, a composition, formulation, or cell culture with a particular initial concentration or mixture, and amounts that differ due to mixing or processing a composition or formulation with a particular initial concentration or mixture. Whether modified by the term “about” the claims appended hereto include equivalents to these quantities. The term “about” further may refer to a range of values that are similar to the stated reference value. In certain embodiments, the term “about” refers to a range of values that fall within 50, 25, 10, 9, 8, 7, 6, 5, 4, 3, 2, 1 percent or less of the stated reference value.

As used herein, the term “polymorphism” and variants thereof refers to the occurrence of two or more alternative genomic sequences or alleles between or among different genomes or individuals. The terms “genetic mutation” or “genetic variation” and variants thereof include polymorphisms.

As used herein the term “single nucleotide polymorphism” (“SNP”) and variants thereof refers to a site of one nucleotide that varies between alleles. A single nucleotide polymorphism (SNP) is a single base change or point mutation but variants also include the so-called “indel” mutations (insertions or deletions of 1 to several up to 75 nucleotides), resulting in genetic variation between individuals. SNPs, which make up about 90% of all human genetic variation, occur every 100 to 300 bases along the 3-billion-base human genome. However, SNPs can occur much more frequently in other organisms like viruses. SNPs can occur in coding or non-coding regions of the genome. A SNP in the coding region may or may not change the amino acid sequence of a protein product. A SNP in a non-coding region can alter promoters or processing sites and may affect gene transcription and/or processing. Knowledge of whether an individual has particular SNPs in a genomic region of interest may provide sufficient information to develop diagnostic, preventive and therapeutic applications for a variety of diseases.

The term “primer” and variants thereof refers to an oligonucleotide that acts as a point of initiation of DNA synthesis in a polymerase chain reaction (PCR). A primer is usually about 10 to about 35 nucleotides in length and hybridizes to a region complementary to the target sequence.

The term “probe” and variants thereof (e.g., detection probe) refers to an oligonucleotide that hybridizes to a target nucleic acid in a PCR reaction. Target sequence refers to a region of nucleic acid that is to be analyzed and comprises the polymorphic site of interest.

The hybridization occurs in such a manner that the probes within a probe set may be modified to form a new, larger molecular entity (e.g., a probe product). The probes herein may hybridize to the nucleic acid regions of interest under stringent conditions. As used herein the term “stringency” is used in reference to the conditions of temperature, ionic strength, and the presence of other compounds such as organic solvents, under which nucleic acid hybridizations are conducted. “Stringency” typically occurs in a range from about Tₘ° C. to about 20° C. to 25° C. below Tₘ. A stringent hybridization may be used to isolate and detect identical polynucleotide sequences or to isolate and detect similar or related polynucleotide sequences. Under “stringent conditions” the nucleotide sequence, in its entirety or portions thereof, will hybridize to its exact complement and closely related sequences. Low stringency conditions comprise conditions equivalent to binding or hybridization at 68° C. in a solution consisting of 5×SSPE (43.8 g/l NaCl, 6.9 g/l NaH₂PO₄·H₂O and 1.85 g/l EDTA, pH adjusted to 7.4 with NaOH), 0.1% SDS, 5×Denhardt's reagent (50×Denhardt's contains per 500 ml: 5 g Ficoll (Type 400), 5 g BSA) and 100 μg/ml denatured salmon sperm DNA followed by washing in a solution comprising 2.0+SSPE, 0.1% SDS at room temperature when a probe of about 100 to about 1000 nucleotides in length is employed. It is well known in the art that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions which promote hybridization under conditions of high stringency (e.g., increasing the temperature of the hybridization and/or wash steps, the use of formamide in the hybridization solution, etc.) are well known in the art. High stringency conditions, when used in reference to nucleic acid hybridization, comprise conditions equivalent to binding or hybridization at 68° C. in a solution consisting of 5+SSPE, 1% SDS, 5×Denhardt's reagent and 100 μg/ml denatured salmon sperm DNA followed by washing in a solution comprising 0.1+SSPE and 0.1% SDS at 68° C. when a probe of about 100 to about 1000 nucleotides in length is employed.

Unless defined otherwise, all technical and scientific terms used herein have the same meaning as commonly understood by those of ordinary skill in the art to which the invention pertains. Although any methods and materials similar or equivalent to those described herein can be used in the practice or testing of the present invention, various embodiments of methods and materials are specifically described herein.

As explained above, KC is the most common corneal ectatic disorder with approximately 6-23.5% of patients carrying a positive family history. (Wheeler, J., Hauser, M. A., Afshari, N. A., Allingham, R. R., Liu, Y. Reproductive Sys Sexual Disord 2012; S:6.) The reported prevalence of KC ranges from 8.8 to 54.4 per 100,000. This variation in prevalence is partly due to the different criteria used to diagnose the disease. (Wheeler, J., Hauser, M. A., Afshari, N. A., Allingham, R. R., Liu, Y. Reproductive Sys Sexual Disord 2012; S:6; and Nowak, D., Gajecka, M. Middle East Afr J Ophthalmol 2011; 18(1):2-6) Many studies exist within the literature that attempt to define the genetic causes of KC. These studies have uncovered numerous possible genetic variants or SNPs that are believed to contribute to the etiology of the disease depending on the experimental parameters.

In general, the work conducted thus far primarily makes use of micro-satellite genotyping and micro-chip technologies (SNP arrays) to interrogate regions of interest within the genome. In comparison, the study described herein utilized Next Gen Sequencing (NGS) technology to identify and to validate genetic variants that contribute to the etiology of the disease. The study involved a whole exome sequencing (WES) approach (ACE Platform™; Personalis Inc., Menlo Park, CA) in which the ˜22,000 genes that comprise the human exome were captured and sequenced; single point mutations or variants including INDELS were identified.

It is recognized that within the human genome there exist various loci harboring gene mutations that contribute to the phenotypical profile of KC. Among those loci documented in the literature are regions mapped to chromosomes 1592.32 and 15q22.33-924.2, 13q32, 16q22.3-q23.1, 3p14-q13, 5q14.3-q21.1, 5921.2 and 5q32-933, 1p36.23-36.21 and 8q13.1-q21.11, 9q34, 14q11.2 and 14q24.3 (sec, for example, Bisceglia L, De Bonis P, Pizzicoli C et al., Invest Ophthalmol Vis Sci. 2009; 50: 1081-1086; Hughes A E, Dash D P, Jackson A J, Frazer D G, Silvestri G, Invest Ophthalmol Vis Sci 2003; 44:5063-5066; Gajecka M, Radhakrishna U, Winters D et al., Invest Ophthalmol Vis Sci 2009; 50:1531-1539; Czugala, M., Karolak, J. A., Nowak, D. A., et. al., European Journal of Human Genetics 2012; 20:389-397; Tyynismaa H, Sistonen P, Tuupanen S et al.; Invest Ophthalmol Vis Sci 2002; 43: 3160-3164; Brancati F, Valente E M, Sarkozy A et al., J Med Genet 2004; 41:188-192; Tang Y G, Rabinowitz Y S, Taylor K D et al., Genet Med 2005; 7: 397-405; Burdon K P, Coster D J, Charlesworth J C et al.; Hum Genet 2008; 124:379-386; Li, X., Rabinowitz, Y. S., Tang, Y. G., Picornell, Y., Taylor, K. D., Hu, M., Yang, H.; Invest Ophthalmol Vis Sci 2006; 47:3791-3795; and Liskova P, Hysi P G, Waseem N, Ebenezer N D, Bhattacharya S S, Tuft S J, Arch Ophthalmol 2010; 128:1191-1195.)

As explained above, these studies mostly utilize micro-satellite genotyping in conjunction with array chip technologies to interrogate regions of interest within the genome.

In addition to the above referenced studies, mutations in the visual system homeobox gene 1 (VSX1) have been identified through the targeted screening of this gene in patients diagnosed with KC. The research conducted on the VSX1 gene so far has not clearly identified a causative agent and in fact, much of the literature presents conflicting results. See, for example, Bisceglia, L., Ciaschetti, M., De Bonis, P., Campo, P. A., Pizzicoli, C., Scala, C., Grifa, M., Ciavarella, P., Delle Noci, N., Vaira, F. et al., Invest Ophthalmol Vis Sci 2005; 46:39-45, Heon, E., Greenberg, A., Kopp, K. K., Rootman, D., Vincent, A. L., Billingsley, G., Priston, M., Dorval, K. M., Chow, R. L., McInnes, R. R. et al., Hum Mol Genet 2002; 11(9):1029-1036, Tang, Y. G., Picornell, Y., Su, X., Li, X., Yang, H. and Rabinowitz, Y. S. Cornea 2008; 27:189-192; Aldave, A. J., Yellore, V. S., Salem, A. K., Yoo, G. L., Rayner, S. A., Yang, H., Tang, G. Y., Piconell, Y., Rabinowitz, Y. S., Invest Ophthalmol Vis Sci 2006; 47(7):2820-2; Tanwar, M., Kumar, M., Nayak, B., Pathak, D., Sharma, N., Titiyal, J. S. and Dada, R., Mol Vis 2010; 16: 2395-2401; Mok, L. W., Back, S. J., Joo, C. K., J Hum Genet 2008; 53:842-849; Jeoung, J. W., Kim, M. K., Park, S. S., Kim, S. Y., Ko, H. S., Won Ryang Wec, W. R., Jin Hak Lec, J. H., Cornea 2012; 31, 7:746-750; Dehkordi, F. A., Rashki, A., Bagheri, N., Chaleshtori, M. H., Memarzadch, E., Salchi, A., Ghatrch, H., Zandi, F., Yazdanpanahi, N., Tabatabaicfar, M. A., Chaleshtori, M. H. Method. Acta Cytologica 2013; 57: 646-651; Sace-Rad, S., Hashemi, H., Miraftab, M., Noori-Daloii, M. R., Chaleshtori, M. H., Raoofian, R., Jafari, F., Greene, W., Fakhraic, G., Rezvan, F., Heidari, M., Mol Vis 2011; 17:3128-3136; Wang, Y., Jin, T., X. Zhang, X., Wei, W., Cui, Y., Geng, T., Liu, Q., Gao, J., Liu, M., Chen, C., Zhang, C., Zhu, X., Ophthalmic Genetics 2013; 34, 3: 160-166; Dash, D. P., S George, S., O'Prey, D., Burns, D., Nabili, S., Donnelly, U., Hughes, A. E., Silvestri, G., Jackson, J., Frazer, D., Hcon, E., Willoughby, C. E., Eye, 2010; 24, 6: 1085-1092.

While much investigative work has been carried out on the possible role of the VSX1 gene in the etiology of KC, this is not the only gene that has been targeted for analysis.

Most prominent among the genes that have been investigated within the literature are the various genes related to the structure of collagen. Collagens are the major protein components of the human cornea, and there exist several types of collagen genes that code for the various collagen proteins. Of interest here are COL4A3 and COL4A4 (Štabuc-Šilih, M., Ravnik-Glavač, M., Glavač, D., Hawlina, M., Stražišar M., Mol Vis 2009; 15:2848-2860; Štabuc-Šilih, M., Stražišar, M., Ravnik Glavač, M., Hawlina, Glavač, D.; Acta Dermatoven APA 2010; 19(2):3-10; Vitart, V., Bencic, G., Hayward, C., Herman, J. S., Huffman J., Campbell, S., Bucan, K., Navarro, P., Gunjaca, G., Marin, J., Zgaga, L., Kolcic, I., Polasek, O., Kirin, M., Hastie, N. D., Wilson, J. F., Rudan, I., Campbell, H., Vatavuk, Z., Fleck, B., Wright, A., Hum Mol Genet 2010; 19(21): 4304-4311) mapped to 2q36.3 (Vitart, V., Bencic, G., Hayward, C., Herman, J. S., Huffman J., Campbell, S., Bucan, K., Navarro, P., Gunjaca, G., Marin, J., Zgaga, L., Kolcic, I., Polasek, O., Kirin, M., Hastic, N. D., Wilson, J. F., Rudan, I., Campbell, H., Vatavuk, Z., Fleck, B., Wright, A., Hum Mol Genet 2010; 19(21): 4304-4311) along with COL4A1 and COL4A2 mapped to the 13q32 locus (Gajecka M, Radhakrishna U, Winters D et al., Invest Ophthalmol Vis Sci 2009; 50:1531-1539; Czugala, M., Karolak, J. A., Nowak, D. A., et. al., European Journal of Human Genetics 2012; 20:389-397; Karolak, J. A., Kulinska, K., Nowak, D. M., Pitarque, J. A., Molinari, A., Rydzanicz, M., Bejjani, B. A., Gajecka, M., Mol Vis 2011; 17:827-843). In reference to the COL4A3 and COL4A4 genes, Štabuc-Šilih et al. in a study published in 2009 identified several SNPs that carried significant p-values. In this study which included 104 unrelated diagnosed patients and 157 healthy blood donors, polymorphism M1327V located at allele 3979 in the COL4A4 gene had a p-value <0.0001 with 134 point mutations out of 208 total alleles for the cases and 132 out of 314 alleles for the controls (Štabuc-Šilih, M., Ravnik-Glavač, M., Glavač, D., Hawlina, M., Stražišar M., Mol Vis 2009; 15:2848-2860). With that said, in a subsequent paper published in 2010, Štabuc-Šilih et al. excludes COL4A3 and COL4A4 from playing a significant role in KC pathogenesis (Štabuc-Šilih, M., Stražišar, M., Ravnik Glavač, M., Hawlina, Glavač, D., Acta Dermatoven APA 2010; 19(2):3-10).

Similarly, Karolak et al. documents findings relating to the COL4A1 and the COL4A2 genes within Ecuadorian families; 23 individuals from one family, 25 affected individuals from other Ecuadorian families, and 64 Ecuadorian control subjects were included in this study (Karolak, J. A., Kulinska, K., Nowak, D. M., Pitarque, J. A., Molinari, A., Rydzanicz, M., Bejjani, B. A., Gajecka, M., Mol Vis 2011; 17:827-843). This study identifies several mutations within the COL4A1 and the COL4A2 genes that were significant. For instance, a polymorphism, Gln1334His found at the 4002 allele on COL4A1 gene was observed more frequently in patients than in healthy individuals in the family where twenty-three individuals (p=0.056) were examined. However, there was no difference in the c. 4002A>C allele distribution between the analyzed affected individuals from the remaining KC families and the Ecuadorian control subjects (p=0.17).

In conjunction with the work described above (Karolak, J. A., Kulinska, K., Nowak, D. M., Pitarque, J. A., Molinari, A., Rydzanicz, M., Bejjani, B. A., Gajecka, M., Mol Vis 2011; 17:827-843), Czugala et. al conducted a study on the same Ecuadorian family group that revealed eight candidate genes other than COL4A1 and COL4A2 (Czugala, M., Karolak, J. A., Nowak, D. A., et. al., European Journal of Human Genetics 2012; 20: 389-397). These genes are MBNL1, IPO5, FARPI, RNF113B, STK24, DOCK9, ZIC5 and ZIC2. Ninety-two sequence variants were identified within these eight genes. At least four of the ninety-two variants referred to in this study show a statistical correlation to the KC phenotype. These genes and the SNPs associated with them are located at the 13q32 locus, but another important aspect of both this study and the work conducted with the COL4A1 and COL4A2 genes is that the results are derived from the genetic analysis primarily of one extended family in Ecuador (Czugala, M., Karolak, J. A., Nowak, D. A., et. al., European Journal of Human Genetics 2012; 20:389-397).

The case studies referenced here were conducted to further elucidate the role of collagen genes and the role they play within the cornea and to investigate the role of the 13q32 locus, a location on the genome that could be an important hotspot within the human genome (Gajecka M, Radhakrishna U, Winters D et al., Invest Ophthalmol Vis Sci 2009; 50:1531-1539; and Czugala, M., Karolak, J. A., Nowak, D. A., et. al., European Journal of Human Genetics 2012; 20:389-397). COL4A3 and COL4A4 genes, which are known to be deregulated in KC patients, are often subjected to chromosomal aberrations, and could also be responsible for a decrease in collagen types I and III, a feature often detected in the disease (Critchfield, J. W., Calandra, A. J., Nesburn, A. B., Kenney, M. C., Exp Eye Res 1988; 46: 953-63; Kenney, M. C., Nesburn, A. B, Burgeson, R. E., Butkowski, R. J., Ljubimov A. V., Cornea 1997; 16:345-51; Meck, K. M., Tuft, S. J., Huang, Y., Gill P. S., Hayes, S., Newton, R. H., Bron, A. J., Invest Ophthalmol Vis Sci 2005; 46:1948-56; Bochert, A., Berlau, J., Koczan, D., Seitz, B., Thiessen, H. J., Guthoff, R. F., Ophthalmologe 2003; 100:545-9; Stachs, O., Bocher, A., Gerber, T., Koczan, D., Thiessen, H. J., Guthoff, R. F., Ophthalmologe 2004; 101:384-9; Pettenati, M. J, Sweatt, A. J., Lantz, P., Stanton, C. A., Reynolds, J., Rao, P. N., Davis, R. M., Hum Genet 1997; 101:26-9).

The search for a genetic link that defines the subset of KC, labeled as familial KC mostly results in the identification of different SNP candidates depending on the family pedigree. For example, the gene, VSX1 was thought to be a primary candidate based on a few isolated family studies (Bisceglia, L., Ciaschetti, M., De Bonis, P., Campo, P. A., Pizzicoli, C., Scala, C., Grifa, M., Ciavarella, P., Delle Noci, N., Vaira, F. et al., Invest Ophthalmol Vis Sci 2005; 46: 39-45; Heon, E., Greenberg, A., Kopp, K. K., Rootman, D., Vincent, A. L., Billingsley, G., Priston, M., Dorval, K. M., Chow, R. L., McInnes, R. R. et al., Hum Mol Genet 2002; 11(9):1029-1036); however, non-family based studies have also been conducted with this gene that involved unrelated individuals of different ethnicities and geographic locations. These studies attempt to identify specific SNPs within the gene that would better define the role of VSX1 (Aldave, A. J., Yellore, V. S., Salem, A. K., Yoo, G. L., Rayner, S. A., Yang, H., Tang, G. Y., Piconell, Y., Rabinowitz, Y. S., Invest Ophthalmol Vis Sci 2006; 47, 7:2820-2; Tanwar, M., Kumar, M., Nayak, B., Pathak, D., Sharma, N., Titiyal, J. S. and Dada, R., Mol Vis 2010; 16: 2395-2401; Mok, L. W., Back, S. J., Joo, C. K., J Hum Genet 2008; 53: 842-849; Jeoung, J. W., Kim, M. K., Park, S. S., Kim, S. Y., Ko, H. S., Won Ryang Wec, W. R., Jin Hak Lec, J. H., Cornea 2012; 31, 7: 746-750; Dehkordi, F. A., Rashki, A., Bagheri, N., Chaleshtori, M. H., Memarzadeh, E., Salchi, A., Ghatrch, H., Zandi, F., Yazdanpanahi, N., Tabatabaicfar, M. A., Chaleshtori, M. H., Acta Cytologica 2013; 57: 646-651, Wang, Y., Jin, T., X. Zhang, X., Wei, W., Cui, Y., Geng, T., Liu, Q., Gao, J., Liu, M., Chen, C., Zhang, C., Zhu, X., Ophthalmic Genetics 2013; 34, 3: 160-166; Dash, D. P., S George, S., O'Prey, D., Burns, D., Nabili, S., Donnelly, U., Hughes, A. E., Silvestri, G., Jackson, J., Frazer, D., Heon, E., Willoughby, C. E., Eye, 2010; 24, 6: 1085-1092). In general, publications resulting from these studies are inconclusive and in fact, the pathogenic role of certain non-synonymous candidate SNPs found within the VSX1 gene has been refuted (Tang, Y. G., Picornell, Y., Su, X., Li, X., Yang, H. and Rabinowitz, Y. S., Cornea 2008; 27: 189-192; Aldave, A. J., Yellore, V. S., Salem, A. K., Yoo, G. L., Rayner, S. A., Yang, H., Tang, G. Y., Piconell, Y., Rabinowitz, Y. S., Invest Ophthalmol Vis Sci 2006; 47(7): 2820-2; Tanwar, M., Kumar, M., Nayak, B., Pathak, D., Sharma, N., Titiyal, J. S. and Dada, R. VSX1 gene analysis in keratoconus. Mol Vis 2010; 16: 2395-2401).

KC with no family associations is the most common form of the disease seen by practicing clinicians (Rabinowitz, Y. S., Ophthalmol Clin N Am. 2003; 16(4): 607-620). With that said, it is likely that familial aggregation has been underreported due to undetected forms of KC. Recent advances in diagnostic techniques such as videokeratography may help better understand whether other forms of the disease are, in actuality, inherited.

The work described above that involves the VSX1 gene (Bisceglia, L., Ciaschetti, M., De Bonis, P., Campo, P. A., Pizzicoli, C., Scala, C., Grifa, M., Ciavarella, P., Delle Noci, N., Vaira, F. et al., Invest Ophthalmol Vis Sci 2005; 46:39-45; Heon, E., Greenberg, A., Kopp, K. K., Rootman, D., Vincent, A. L., Billingsley, G., Priston, M., Dorval, K. M., Chow, R. L., McInnes, R. R. et al.; Hum Mol Genet 2002; 11(9): 1029-1036; Tang, Y. G., Picornell, Y., Su, X., Li, X., Yang, H. and Rabinowitz, Y. S. Cornea 2008; 27:189-192; Aldave, A. J., Yellore, V. S., Salem, A. K., Yoo, G. L., Rayner, S. A., Yang, H., Tang, G. Y., Piconell, Y., Rabinowitz, Y. S. Invest Ophthalmol Vis Sci 2006; 47(7): 2820-2; Tanwar, M., Kumar, M., Nayak, B., Pathak, D., Sharma, N., Titiyal, J. S. and Dada, R. Mol Vis 2010; 16: 2395-2401; Mok, L. W., Back, S. J., Joo, C. K. J Hum Genet 2008; 53: 842-849; Jeoung, J. W., Kim, M. K., Park, S. S., Kim, S. Y., Ko, H. S., Won Ryang Wee, W. R., Jin Hak Lec, J. H. VSX1 Gene and Keratoconus: Genetic Analysis in Korean Patients Cornea 2012; 31(7): 746-750; Dehkordi, F. A., Rashki, A., Bagheri, N., Chaleshtori, M. H., Memarzadch, E., Salchi, A., Ghatrch, H., Zandi, F., Yazdanpanahi, N., Tabatabaiefar, M. A., Chaleshtori, M. H., Acta Cytologica 2013; 57: 646-651; Sace-Rad, S., Hashemi, H., Miraftab, M., Noori-Daloii, M. R., Chaleshtori, M. H., Raoofian, R., Jafari, F., Greene, W., Fakhraic, G., Rezvan, F., Heidari, M. Mol Vis 2011; 17: 3128-3136; Wang, Y., Jin, T., X. Zhang, X., Wei, W., Cui, Y., Geng, T., Liu, Q., Gao, J., Liu, M., Chen, C., Zhang, C., Zhu, X., Common single nucleotide polymorphisms and keratoconus in the Han Chinese population. Ophthalmic Genetics 2013; 34(3): 160-166) and the various COL genes (Štabuc-Šilih, M., Ravnik-Glavač, M., Glavač, D., Hawlina, M., Stražišar M., Mol Vis 2009; 15:2848-2860; Štabuc-Šilih, M., Stražišar, M., Ravnik Glavač, M., Hawlina, Glavač, D. Acta Dermatoven APA 2010; 19(2):3-10; Karolak, J. A., Kulinska, K., Nowak, D. M., Pitarque, J. A., Molinari, A., Rydzanicz, M., Bejjani, B. A., Gajecka, M., Mol Vis 2011; 17: 827-843; Critchfield, J. W., Calandra, A. J., Nesburn, A. B., Kenney, M. C., Exp Eye Res 1988; 46:953-63; Kenney, M. C., Nesburn, A. B, Burgeson, R. E., Butkowski, R. J., Ljubimov A. V., Cornea 1997; 16: 345-51; Meck, K. M., Tuft, S. J., Huang, Y., Gill P. S., Hayes, S., Newton, R. H., Bron, A. J., Invest Ophthalmol Vis Sci 2005; 46:1948-56; Bochert, A., Berlau, J., Koczan, D., Seitz, B., Thiessen, H. J., Guthoff, R. F., Ophthalmologe 2003; 100:545-9; Stachs, O., Bocher, A., Gerber, T., Koczan, D., Thiessen, H. J., Guthoff, R. F., Ophthalmologe 2004; 101: 384-9; Pettenati, M. J, Sweatt, A. J., Lantz, P., Stanton, C. A., Reynolds, J., Rao, P. N., Davis, R. M., Hum Genet 1997; 101:26-9; Li, X., Bykhovskaya, Y., Caiado Canedo, A. L., Haritunians, T., Siscovick, D., Anthony J. Aldave, A. J., Szczotka-Flynn, L., Iyengar, S. K., Rotter, J. I., Taylor, K. D., Yaron S. Rabinowitz, Y. S., Invest Ophthalmol Vis Sci 2013; 54: 2696-2704) are just a few examples where mutations within genes may be contributing to the phenotype of the disease. These studies primarily focus on the structure and function of one or two genes of interest and in doing so overlook the possibility of other gene mutations within the genome that may contribute to the etiology of the disease. Much of the literature stipulates that genetically, KC is a complex disease (Bisceglia L, De Bonis P, Pizzicoli C et al., Invest Ophthalmol Vis Sci 2009; 50:1081-1086; Tang Y G, Rabinowitz Y S, Taylor K D et al., Genet Med 2005; 7:397-405; Li, X., Rabinowitz, Y. S., Tang, Y. G., Picornell, Y., Taylor, K. D., Hu, M., Yang, H., Invest Ophthalmol Vis Sci 2006; 47:3791-3795; Liskova P, Hysi P G, Waseem N, Ebenezer N D, Arch Ophthalmol 2010; 128:1191-1195; Wheeler, J., Hauser, M. A., Afshari, N. A., Allingham, R. R., Liu, Y., Reproductive Sys Sexual Disord 2012; S:6; Nowak, D., Gajecka, M., Middle East Afr J Ophthalmol 2011; 18(1):2-6; Burdon, K. P. and Vincent, A. L. Clin Exp Optom 2013; 96: 146-154), implicating multiple mutations within more than one gene. HGF and LOX genes harbor SNPs that have been identified as significant in patients diagnosed with KC (Burdon, K. P., Macgregor, S., Bykhovskaya, Y., Javadiyan, S., Li, X., Laurie, K. J., Muszynska, D., Lindsay, R., Lechner, J., Haritunians, T., Henders, A. K., Dash, D., Siscovick, D., Anand, S., Aldave, A., Coster, D. J., Szczotka-Flynn, L., Mills, R. A., Iyengar, S. K., Taylor, K. D., Phillips, T., Grant W. Montgomery, G. W., Rotter, J. I., Hewitt, A. W., Sharma, S., Rabinowitz, Y. S., Willoughby, C., Craig, J. E., Invest Ophthalmol Vis Sci 2011; 52(11): 8514-8519; Sahebjada, S., Schache, M., Richardson, A. J., Snibson, G., Daniell, M., Baird, P. N., PLOS ONE 2014; 9, 1; Dudakova, L., Palos, M., Jirsova, K., Stranecky, V., Krepelova, A., Hysi P. G., Liskova, P., Eur J Hum Genet. 2015; Bykhovskaya, Y., Li, X., Epifantseva, I., Haritunians, T., Siscovick, D., Aldave, A., Szczotka-Flynn, L., Iyengar, S. K., Taylor, K. D., Rotter, J. I., Rabinowitz, Y. S., Invest Ophthalmol Vis Sci; 2012; 53(7): 4152-4157; Hao XD1, Chen P, Chen Z L, Li S X, Wang Y., Ophthalmic Genet. 2015; 36(2): 132-136).

The HGF gene is known to be expressed in the cornea by all three cellular layers (Wilson S E, Walker J W, Chwang E L, He Y G., Invest Ophthalmol Vis Sci. 1993; 34, 8: 2544-2561). The protein is also produced in the lacrimal glands, and HGF expression in corneal keratinocytes is upregulated in response to corneal injury suggesting its involvement in the epithelial wound healing process (Burdon, K. P., Macgregor, S., Bykhovskaya, Y., Javadiyan, S., Li, X., Laurie, K. J., Muszynska, D., Lindsay, R., Lechner, J., Haritunians, T., Henders, A. K., Dash, D., Siscovick, D., Anand, S., Aldave, A., Coster, D. J., Szczotka-Flynn, L., Mills, R. A., Iyengar, S. K., Taylor, K. D., Phillips, T., Grant W. Montgomery, G. W., Rotter, J. I., Hewitt, A. W., Sharma, S., Rabinowitz, Y. S., Willoughby, C., Craig, J. E., Invest Ophthalmol Vis Sci 2011; 52(11): 8514-8519; Li Q, Weng J, Mohan R R, et al., Invest Ophthalmol Vis Sci. 1996; 37(5): 727-739). Furthermore, certain SNPs associated with the HGF gene have been correlated to hypermetropia and myopia (Yanovitch, T., Li, Y. J., Metlapally, R., Abbott, D., Tran Viet, K. N., Young, T. L., Mol Vis 2009; 15: 1028-1035; Veerappan, S., Pertile, K. K., Islam, A. F., Schäche, M., Chen, C. Y., Mitchell, P., Dirani, M., Baird, P. N., Ophthalmology 2010; 117(2): 239-245) along with primary angle closure glaucoma (PACG) (Awadalla, M. S., Thapa, S. S., Burdon, K. P., Hewitt, A. W., Craig, J. E., Mol Vis 2011; 17: 2248-2254).

A subset of the SNPs found to be associated with these various eye conditions were also found in the genomes of KC patients (Burdon, K. P., Macgregor, S., Bykhovskaya, Y., Javadiyan, S., Li, X., Laurie, K. J., Muszynska, D., Lindsay, R., Lechner, J., Haritunians, T., Henders, A. K., Dash, D., Siscovick, D., Anand, S., Aldave, A., Coster, D. J., Szczotka-Flynn, L., Mills, R. A., Iyengar, S. K., Taylor, K. D., Phillips, T., Grant W. Montgomery, G. W., Rotter, J. I., Hewitt, A. W., Sharma, S., Rabinowitz, Y. S., Willoughby, C., Craig, J. E., Invest Ophthalmol Vis Sci 2011; 52(11): 8514-8519).

Regarding the role of the HGF protein in the eye, Burdon et al. states, “The refractive power of the eye is determined at least in part by the shape of the cornea, which is severely altered in KC, thus suggesting overlap between the genetic determinants of these complex ophthalmic conditions” (Burdon, K. P., Macgregor, S., Bykhovskaya, Y., Javadiyan, S., Li, X., Laurie, K. J., Muszynska, D., Lindsay, R., Lechner, J., Haritunians, T., Henders, A. K., Dash, D., Siscovick, D., Anand, S., Aldave, A., Coster, D. J., Szczotka-Flynn, L., Mills, R. A., Iyengar, S. K., Taylor, K. D., Phillips, T., Grant W. Montgomery, G. W., Rotter, J. I., Hewitt, A. W., Sharma, S., Rabinowitz, Y. S., Willoughby, C., Craig, J. E., Invest Ophthalmol Vis Sci 2011; 52(11): 8514-8519). There exist at least two other studies published within the literature that provide verification that the HGF gene is associated with KC (Sahebjada, S., Schache, M., Richardson, A. J., Snibson, G., Daniell, M., Baird, P. N. PLOS ONE 2014, 9(1); Dudakova, L., Palos, M., Jirsova, K., Stranecky, V., Krepelova, A., Hysi P. G., Liskova, P., Eur J Hum Genet. 2015).

LOX encodes an enzyme that initiates the crosslinking of collagens and elastin in a variety of tissues including the cornea (Hamalainen, E. R, Jones, T. A., Sheer, D., Taskinen, K., Pihlajanemi, T., Kivirikko, K. I. Genomics. 1991; 11:508-516). Li et al. carried out a genome-wide linkage scan that mapped several loci to KC including the 5q23.2 locus where the LOX gene is located (Li, X., Rabinowitz, Y. S., Tang, Y. G., Picornell, Y., Taylor, K. D., Hu, M., Yang, H. Invest Ophthalmol Vis Sci 2006; 47:3791-3795). In addition LOX expression levels were found to be upregulated in a study that analyzed KC epithelium on microarrays (Nielsen, K., Birkenkamp-Demtroder, K., Ehlers, N., Orntoft, T. F. Invest Ophthalmol Vis Sci. 2003; 44: 2466-2476). Bykhovskaya et al. in a study that involved two independent panels of patients with KC and controls and KC families found at least four SNPs within this gene that are associated with KC (Bykhovskaya, Y., Li, X., Epifantseva, I., Haritunians, T., Siscovick, D., Aldave, A., Szczotka-Flynn, L., Iyengar, S. K., Taylor, K. D., Rotter, J. I., Rabinowitz, Y. S. Invest Ophthalmol Vis Sci; 2012; 53, 7: 4152-4157). This work was duplicated in a group that found the rs2956540 SNP to be associated with KC in a population of European descent (Dudakova, L., Palos, M., Jirsova, K., Stranecky, V., Krepelova, A., Hysi P. G., Liskova, P., Eur J Hum Genet. 2015) and again in a study conducted on a Han Chinese population (Hao XD1, Chen P, Chen Z L, Li S X, Wang Y., Ophthalmic Genet. 2015; 36, 2: 132-136).

Since riboflavin/ultraviolet-a-induced corneal collagen cross-linking (CXL) has become a prevalent form of treatment for the KC patient (Ashwin, P. T., McDonnell, P. J. Collagen cross-linkage: a comprehensive review and directions for future research. Br J Ophthalmol. 2010; 94: 965-970), there is interest in a gene such as LOX which encodes for a molecular pathway that lead to collagen cross-linking in the cornea. It is believed that knowing the genotype of the LOX gene within the KC patient may have implications and provide insight into the outcome of CXL treatment (Bykhovskaya, Y., Li, X., Epifantseva, I., Haritunians, T., Siscovick, D., Aldave, A., Szczotka-Flynn, L., Iyengar, S. K., Taylor, K. D., Rotter, J. I., Rabinowitz, Y. S. Invest Ophthalmol Vis Sci; 2012; 53, 7: 4152-4157).

In one aspect, the disclosure provides methods for isolating genomic samples to identify and validate single nucleotide polymorphism detection. In some embodiments, the genomic samples may be selected from the group consisting of isolated cells, whole blood, serum, plasma, urine, saliva, sweat, fecal matter, and tears.

In some embodiments, the genomic sample is plasma or serum, and the method further comprises isolating the plasma or serum from a blood sample of the subject.

In some embodiments, the method includes providing a sample of cells from a subject. In some embodiments, the cells are collected by contacting a cellular surface of a subject with a substrate capable of reversibly immobilizing the cells onto a substrate.

The disclosed methods are applicable to a variety of cell types obtained from a variety of samples. In some embodiments, the cell type for use with the disclosed methods include but is not limited to epithelial cells, endothelial cells, connective tissue cells, skeletal muscle cells, endocrine cells, cardiac cells, urinary cells, melanocytes, keratinocytes, blood cells, white blood cells, buffy coat, hair cells (including, e.g., hair root cells) and/or salival cells. In some embodiments, the cells are epithelial cells. In some embodiments, the cells are subcapsular-perivascular (epithelial type 1); pale (epithelial type 2); intermediate (epithelial type 3); dark (epithelial type 4); undifferentiated (epithelial type 5); and large-medullary (epithelial type 6). In some embodiments, the cells are buccal epithelial cells (e.g., epithelial cells collected using a buccal swab). In some embodiments, the sample of cells used in the disclosed methods include any combination of the above identified cell types.

In some embodiments, the method includes providing a sample of cells from a subject. In some embodiments, the cells provided are buccal epithelial cells.

The cell sample is collected by any of a variety of methods which allow for reversible binding of the subjects cells to the substrate. In some embodiments, the substrate is employed in a physical interaction with the sample containing the subject's cells in order to reversibly bind the cells to the substrate. In some embodiments, the substrate is employed in a physical interaction with the body of the subject directly in order to reversibly bind the cells to the substrate. In some embodiments, the sample is a buccal cell sample and the sample of buccal cells is collected by contacting a buccal membrane of the subject (e.g., the inside of their check) with a substrate capable of reversibly immobilizing cells that are dislodged from the membrane. In such embodiments, the swab is rubbed against the inside of the subject's cheek with a force equivalent to brushing a person's teeth (e.g., a light amount of force or pressure). Any method which would allow the subject's cells to be reversibly bound to the substrate is contemplated for use with the disclosed methods.

In some embodiments, the sample is advantageously collected in a non-invasive manner. As such sample collection is accomplished anywhere and by almost anyone. For example, in some embodiments, the sample is collected at a physician's office, at a subject's home, or at a facility where a medical procedure is performed or to be performed. In some embodiments the subject, the subject's doctor, nurses or a physician's assistant or other clinical personnel collects the sample.

In some embodiments the substrate is made of any of a variety of materials to which cells are reversibly bound. Exemplary substrates include those made of rayon, cotton, silica, an elastomer, a shellac, amber, a natural or synthetic rubber, cellulose, BAKELITE, NYLON, a polystyrene, a polyethylene, a polypropylene, a polyacrylonitrile, or other materials or combinations thereof. In some embodiments, the substrate is a swab having a rayon tip or a cotton tip.

In some embodiments, the substrate containing the sample is freeze-thawed one or more times (e.g., after being frozen, the substrate containing the sample is thawed, used according to the present methods and re-frozen) and or used in the present methods.

In another aspect, a variety of lysis solutions have been described and are known to those of skill in the art. Any of these well-known lysis solutions can be employed with the present methods in order to isolate nucleic acids from a sample. Exemplary lysis solutions include those commercially available, such as those sold by INVITROGEN®, QIAGEN®, LIFE TECHNOLOGIES® and other manufacturers, as well as those which can be generated by one of skill in a laboratory setting. Lysis buffers have also been well described and a variety of lysis buffers can find use with the disclosed methods, including for example those described in Molecular Cloning (three volume set, Cold Spring Harbor Laboratory Press, 2012) and Current Protocols (Genetics and Genomics; Molecular Biology; 2003-2013), both of which are incorporated herein by reference for all purposes.

Cell lysis is a commonly practiced method for the recovery of nucleic acids from within cells. In many cases, the cells are contacted with a lysis solution, commonly an alkaline solution comprising a detergent, or a solution of a lysis enzyme. Such lysis solutions typically contain salts, detergents and buffering agents, as well as other agents that one of skill would understand to use. After full and/or partial lysis, the nucleic acids are recovered from the lysis solution.

In some embodiments, cells are resuspended in an aqueous buffer, with a pH in the range of from about pH 4 to about 10, about 5 to about 9, about 6 to about 8 or about 7 to about 9.

In some embodiments, the buffer salt concentration is from about 10 mM to about 200 mM, about 10 mM to about 100 mM or about 20 mM to about 80 mM.

In some embodiments, the buffer further comprises chelating agents such as ethylenediaminetetraacetic acid (EDTA) or ethylene glycol tetraacetic acid (EGTA).

In some embodiments, the lysis solution further comprises other compounds to assist with nucleic acid release from cells such as polyols, including for example but not limited to sucrose, as well as sugar alcohols such as maltitol, sorbitol, xylitol, erythritol, and/or isomalt. In some embodiments, polyols are in the range of from about 2% to about 15% w/w, or about 5% to about 15% w/w or about 5% to about 10% w/w.

In some embodiments, the lysis solutions further comprises surfactants, such as for example but not limited to Triton X-100, SDS, CTAB, X-114, CHAPS, DOC, and/or NP-40. In some embodiments such surfactants are in the range of from about 1% to about 5% w/w, about 1% to about 4% w/w, or about 1% to about 3% w/w.

In embodiments, the lysis solution further comprises chaotropes, such as for example but not limited to urea, sodium dodecyl sulfate and/or thiourea. In some embodiments, the chaotrope is used at a concentration in the range of from about 0.5 M to 8 M, about 1 M to about 6 M, about 2 M to about 6 M or about 1 M to 3 M.

In some embodiments, the lysis solution further comprises one or more additional lysis reagents and such lysis reagents are well known in the art. In some embodiments, such lysis reagents include cell wall lytic enzymes, such as for example but not limited to lysozyme. In some embodiments, lysis reagents comprise alkaline detergent solutions, such as 0.1 aqueous sodium hydroxide containing 0.5% sodium dodecyl sulphate.

In some embodiments, the lysis solution further comprises aqueous sugar solutions, such as sucrose solution and chelating agents such as EDTA, for example the STET buffer. In certain embodiments, the lysis reagent is prepared by mixing the cell suspension with an equal volume of lysis solution having twice the desired concentration (for example 0.2 sodium hydroxide, 1.0% sodium dodecyl sulphate).

In some embodiments, after the desired extent of lysis has been achieved, the mixture comprising lysis solution and lysed cells is contacted with a neutralizing or quenching reagent to adjust the conditions such that the lysis reagent does not adversely affect the desired product. In some embodiments, the pH is adjusted to a pH of from about 5 to about 9, about 6 to about 8, about 5 to about 7, about 6 to about 7 or about 6.5 to 7.5 to minimize and/or prevent degradation of the cell contents, including for example but not limited to the nucleic acids. In some embodiments, when the lysis reagent comprises an alkaline solution, the neutralizing reagent comprises an acidic buffer, for example an alkali metal acetate/acetic acid buffer. In some embodiments, lysis conditions, such as temperature and composition of the lysis reagent are chosen such that lysis is substantially completed while minimizing degradation of the desired product, including for example but not limited to nucleic acids.

Any combination of the above can be employed by one of skill, as well as combined with other known and routine methods, and such combinations are contemplated by the present invention.

In another aspect, the nucleic acids, including for example but not limited to genomic DNA, are isolated from lysis buffer prior to performing subsequent analysis. In some embodiments, the nucleic acids are isolated from the lysis buffer prior to the performance of additional analyses, such as for example but not limited to real-time PCR analyses. Any of a variety of methods useful in the isolation of small quantities of nucleic acids are used by various embodiments of the disclosed methods. These include but are not limited to precipitation, gel filtration, density gradients and solid phase binding. Such methods have also been described in for example, Molecular Cloning (three volume set, Cold Spring Harbor Laboratory Press, 2012) and Current Protocols (Genetics and Genomics; Molecular Biology; 2003-2013), incorporated herein by reference for all purposes.

Nucleic Acid precipitation is a well know method for isolation that is known by those of skill in the art. A variety of solid phase binding methods are also known in the art including but not limited to solid phase binding methods that make use of solid phases in the form of beads (e.g., silica, magnetic), columns, membranes or any of a variety other physical forms known in the art. In some embodiments, solid phases used in the disclosed methods reversibly bind nucleic acids. Examples of such solid phases include so-called “mixed-bed” solid phases are mixtures of at least two different solid phases, each of which has a capacity to nucleic acids under different solution conditions, and the ability and/or capacity to release the nucleic acid under different conditions; such as those described in US Patent Application No. 2002/0001812, incorporated by reference herein in its entirety for all purposes. Solid phase affinity for nucleic acids according to the disclosed methods can be through any one of a number of means typically used to bind a solute to a substrate. Examples of such means include but are not limited to, ionic interactions (e.g., anion-exchange chromatography) and hydrophobic interactions (e.g., reversed-phase chromatography), pH differentials and changes, salt differentials and changes (e.g., concentration changes, use of chaotropic salts/agents). Exemplary pH based solid phases include but are not limited to those used in the INVITROGEN ChargeSwitch Normalized Buccal Kit magnetic beads, to which bind nucleic acids at low pH (<6.5) and releases nucleic acids at high pH (>8.5) and mono-amino-N-aminocthyl (MANAE) which binds nucleic acids at a pH of less than 7.5 and release nucleic acids at a pH of greater than 8. Exemplary ion exchange based substrates include but are not limited to DEA-SEPHAROSE™, Q-SEPHAROSE™, and DEAE-SEPHADEX™ from PHARMACIA (Piscataway, N.J.), DOWEX® I from The Dow Chemical Company (Midland, Mich.), AMBERLITE® from Rohm & Haas (Philadelphia, Pa.), DUOLITE® from Duolite International, In. (Cleveland, Ohio), DIALON TI and DIALON TII.

Any individual method is contemplated for use alone or in combination with other methods, and such useful combination are well known and appreciated by those of skill in the art.

In another aspect, the disclosed methods are used to isolate nucleic acids, such as genomic DNA (gDNA) for a variety of nucleic acid analyses, including genomic analyses. In some embodiments, such analysis includes detection of variety of genetic mutations, which include but are not limited to deletions, insertions, transitions and transversions. In some embodiments, the mutation is a single-nucleotide polymorphism (SNP).

A variety of methods for analyzing such isolated nucleic acids, for example but not limited to genomic DNA (gDNA) are known in the art and include nucleic acid sequencing methods (including Next Generation Sequencing methods), PCR methods (including real-time PCR analysis, microarray analysis, hybridization analysis) as well as any other nucleic acid sequence analysis methods that are known in the art, which include a variety of other methods where nucleic acid compositions are analyzed and which are known to those of skill in the art. See, for example, Molecular Cloning (three volume set, Cold Spring Harbor Laboratory Press, 2012) and Current Protocols (Genetics and Genomics; Molecular Biology; 2003-2013).

In one aspect, the SNP described herein may be detected by sequencing. For example, High-throughput or Next Generation Sequencing (NGS) represents an attractive option for detecting mutations within a gene. Distinct from PCR, microarrays, high-resolution melting and mass spectrometry, which all indirectly infer sequence content, NGS directly ascertains the identity of each base and the order in which they fall within a gene. The newest platforms on the market have the capacity to cover an exonic region 10,000 times over, meaning the content of each base position in the sequence is measured thousands of different times. This high level of coverage ensures that the consensus sequence is extremely accurate and enables the detection of rare variants within a heterogeneous sample. For example, in a sample extracted from formalin-fixed, paraffin-embedded (FFPE) tissue, often a mutation of interest is only present at a frequency of 1%. When this sample is sequenced at 10,000λ coverage, then even the rare allele, comprising only 1% of the sample, is uniquely measured 100 times over. Thus, NGS provides reliably accurate results with very high sensitivity, making it ideal for clinical diagnostic testing of FFPEs and other mixed samples.

Examples of sequencing techniques, often referred to as Next Generation Sequencing (NGS) techniques include, but are not limited to Sequencing by Synthesis (SBS), Massively Parallel Signature Sequencing (MPSS), Polony sequencing, pyrosequencing, Reversible dye-terminator sequencing, SOLID sequencing, Ion semiconductor sequencing, DNA nanoball sequencing, Helioscope single molecule sequencing, Single molecule real time (SMRT) sequencing, Single molecule real time (RNAP) sequencing, and Nanopore DNA sequencing.

MPSS was a bead-based method that used a complex approach of adapter ligation followed by adapter decoding, reading the sequence in increments of four nucleotides; this method made it susceptible to sequence-specific bias or loss of specific sequences.

Polony sequencing, combined an in vitro paired-tag library with emulsion PCR, an automated microscope, and ligation-based sequencing chemistry to sequence an E. coli genome at an accuracy of >99.9999% and a cost approximately 1/10 that of Sanger sequencing.

A parallelized version of pyrosequencing, the method amplifies DNA inside water droplets in an oil solution (emulsion PCR), with each droplet containing a single DNA template attached to a single primer-coated bead that then forms a clonal colony. The sequencing machine contains many picolitre-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLID on the other.

SBS is a sequencing technology based on reversible dye-terminators. DNA molecules are first attached to primers on a flowcell and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

SOLID technology employs sequencing by ligation. Here, a pool of all possible oligonucleotides of a fixed length are labeled according to the sequenced position.

Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. The result is sequences of quantities and lengths comparable to Illumina sequencing.

Ion semiconductor sequencing is based on using standard sequencing chemistry, but with a novel, semiconductor based detection system. This method of sequencing is based on the detection of hydrogen ions that are released during the polymerization of DNA, as opposed to the optical methods used in other sequencing systems. A micro well containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

DNA nanoball sequencing is a type of high throughput sequencing technology used to determine the entire genomic sequence of an organism. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run.

Helicos Biosciences Corporation's single-molecule sequencing uses DNA fragments with added polyA tail adapters, which are attached to the flow cell surface. The next steps involve extension-based sequencing with cyclic washes of the flow cell with fluorescently labeled nucleotides (one nucleotide type at a time, as with the Sanger method). The reads are performed by the Helioscope sequencer.

Single molecule real time (SMRT) sequencing is based on the SBS approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand.

Single molecule real time sequencing based on RNA polymerase (RNAP), which is attached to a polystyrene bead, with distal end of sequenced DNA is attached to another bead, with both beads being placed in optical traps. RNAP motion during transcription brings the beads in closer and their relative distance changes, which can then be recorded at a single nucleotide resolution. The sequence is deduced based on the four readouts with lowered concentrations of each of the four nucleotide types (similarly to Sangers method).

Nanopore sequencing is based on the readout of electrical signal occurring at nucleotides passing by alpha-hemolysin pores covalently bound with cyclodextrin. The DNA passing through the nanopore changes its ion current. This change is dependent on the shape, size and length of the DNA sequence. Each type of the nucleotide blocks the ion flow through the pore for a different period of time.

VisiGen Biotechnologies uses a specially engineered DNA polymerase. This polymerase acts as a sensor—having incorporated a donor fluorescent dye by its active centre. This donor dye acts by FRET (fluorescent resonant energy transfer), inducing fluorescence of differently labeled nucleotides. This approach allows reads performed at the speed at which polymerase incorporates nucleotides into the sequence (several hundred per second). The nucleotide fluorochrome is released after the incorporation into the DNA strand.

Mass spectrometry may be used to determine mass differences between DNA fragments produced in chain-termination reactions.

SBS technology is capable of overcoming the limitations of existing pyrosequencing based NGS platforms.

Such technologies rely on complex enzymatic cascades for read out, are unreliable for the accurate determination of the number of nucleotides in homopolymeric regions and require excessive amounts of time to run individual nucleotides across growing DNA strands. The SBS NGS platform uses a direct sequencing approach to produce a sequencing strategy with very a high precision, rapid pace and low cost.

One exemplary SBS sequencing is initialized by fragmenting of the template DNA into fragments, amplification, annealing of DNA sequencing primers, and, for example, finally affixing as a high-density array of spots onto a glass chip. The array of DNA fragments are sequenced by extending each fragment with modified nucleotides containing cleavable chemical moicties linked to fluorescent dyes capable of discriminating all four possible nucleotides. The array is scanned continuously by a high-resolution electronic camera (Measure) to determine the fluorescent intensity of each base (A, C, G or T) that was newly incorporated into the extended DNA fragment. After the incorporation of each modified base the array is exposed to cleavage chemistry to break off the fluorescent dye and end cap allowing additional bases to be added. The process is then repeated until the fragment is completely sequenced or maximal read length has been achieved.

In another aspect, real-time PCR is used in detecting gene mutations, including for example but not limited to SNPs. In some embodiments, detection of SNPs in specific gene candidates is performed using real-time PCR, based on the use of intramolecular quenching of a fluorescent molecule by use of a tethered quenching moiety. Thus, according to exemplary embodiments, real-time PCR methods also include the use of molecular beacon technology. The molecular beacon technology utilizes hairpin-shaped molecules with an internally-quenched fluorophore whose fluorescence is restored by binding to a DNA target of interest (Sec, e.g., Kramer, R. et al. Nat. Biotechnol. 14:303-308, 1996). In some embodiments, increased binding of the molecular beacon probe to the accumulating PCR product is used to specifically detect SNPs present in genomic DNA.

For the design of Real-Time PCR assays, several parts are coordinated, including the DNA fragment that is flanked by the two primers and subsequently amplified, often referred to as the amplicon, the two primers and the detection probe or probes to be used.

In some embodiments, a SNP site in a sample from the subject may be amplified by the amplification methods described herein or any other amplification methods known in the art. The nucleic acids in a sample may or may not be amplified prior to contacting the SNP site with a probe described herein, using a universal amplification method (e.g., whole genome amplification and whole genome PCR).

Real-time PCR relies on the visual emission of fluorescent dyes conjugated to short polynucleotides (termed “detection probes”) that associate with genomic alleles in a sequence-specific fashion or on fluorescent molecules that intercalate into double stranded DNA referred to as quantitative or qPCR. Real-time PCR probes differing by a single nucleotide can be differentiated in a real-time PCR assay by the conjugation and detection of probes that fluoresce at different wavelengths. Real-Time PCR finds use in detection applications (diagnostic applications), quantification applications and genotyping applications.

Several related methods for performing real-time PCR are disclosed in the art, including assays that rely on TAQMAN® probes (U.S. Pat. Nos. 5,210,015 and 5,487,972, and Lec et al., Nucleic Acids Res. 21:3761-6, 1993), molecular beacon probes (U.S. Pat. Nos. 5,925,517 and 6, 103, 476, and Tyagi and Kramer, Nat. Biotechnol. 14:303-8, 1996), self-probing amplicons (scorpions) (U.S. Pat. No. 6,326,145, and Whitcombe et al., Nat. Biotechnol. 17:804-7, 1999), Amplisensor (Chen et al., Appl. Environ. Microbiol. 64:4210-6, 1998), Amplifluor (U.S. Pat. No. 6,117,635, and Nazarenko et al., Nucleic Acids Res. 25:2516-21, 1997, displacement hybridization probes (Li et al., Nucleic Acids Res. 30:E5, 2002), DzyNA-PCR (Todd et al., Clin. Chem. 46:625-30, 2000), fluorescent restriction enzyme detection (Cairns et al., Biochem. Biophys. Res. Commun. 318:684-90, 2004) and adjacent hybridization probes (U.S. Pat. No. 6,174,670 and Wittwer et al., Biotechniques 22:130-1, 134-8, 1997).

One of the many suitable genotyping procedures is the TAQMAN® allelic discrimination assay. In some instances of this assay, an oligonucleotide probe labeled with a fluorescent reporter dye at the 5′ end of the probe and a quencher dye at the 3′ end of the probe is utilized. The proximity of the quencher to the intact probe maintains a low fluorescence for the reporter. During the PCR reaction, the 5′ nuclease activity of DNA polymerase cleaves the probe, and separates the dye and quencher. This results in an increase in fluorescence of the reporter. Accumulation of PCR product is detected directly by monitoring the increase in fluorescence of the reporter dye. The 5′ nuclease activity of DNA polymerase cleaves the probe between the reporter and the quencher only if the probe hybridizes to the target and is amplified during PCR. The probe is designed to straddle a target SNP position and hybridize to the nucleic acid molecule only if a particular SNP allele is present.

Real-time PCR methods include a variety of steps or cycles as part of the methods for amplification. These cycles include denaturing double-stranded nucleic acids, annealing a forward primer, a reverse primer and a detection probe to the target genomic DNA sequence and synthesizing (i.e., replicating) second-strand DNA from the annealed forward primer and the reverse primer. This three step process is referred to herein as a cycle.

In some embodiments, about 10, 15, 20, 25, 30, 35, 40, 45, 50, 55, or 60 cycles are employed. In some embodiments, about 10 to about 60 cycles, about 20 to about 50 or about 30 to about 40 cycles are employed. In some embodiments, 40 cycles are employed.

In some embodiments, the denaturing double-stranded nucleic acids step occurs at a temperature of about 80° C. to 100° ° C., about 85° C. to about 99° C., about 90° C. to about 95° C. for about 1 second to about 5 seconds, about 2 seconds to about 5 seconds, or about 3 seconds to about 4 seconds. In some embodiments, the denaturing double-stranded nucleic acids step occurs at a temperature of 95° C. for about 3 seconds.

In some embodiments, the annealing a forward primer, a reverse primer and a detection probe to the target genomic DNA sequence step occurs at about 40° C. to about 80° ° C., about 50° ° C. to about 70° C., about 55° ° C. to about 65° C. for about 15 seconds to about 45 seconds, about 20 seconds to about 40 seconds, about 25 seconds to about 35 seconds. In some embodiments, the annealing a forward primer, a reverse primer and a detection probe to the target genomic DNA sequence step occurs at about 60° C. for about 30 seconds.

In some embodiments, the synthesizing (i.e., replicating) second-strand DNA from the annealed forward primer and the reverse primer occurs at about 40° C. to about 80° C., about 50° ° C. to about 70° C., about 55° ° C. to about 65° C. for about 15 seconds to about 45 seconds, about 20 seconds to about 40 seconds, about 25 seconds to about 35 seconds. In some embodiments, the annealing a forward primer, a reverse primer and a detection probe to the target genomic DNA sequence step occurs at about 60° ° C. for about 30 seconds.

In some embodiments, it was found that about 1 μL, about 2 μL, about 3 μL, about 4 μL or about 5 μL of a genomic DNA sample prepared according to the present methods described herein, are combined with only about 0.05 μL, about 0.10 μL about 0.15 μL, about 0.20 μL, about 0.25 μL or about 0.25 μL of a 30×, 35×, 40×, 45×, 50× or 100× real-time PCR assay mix and distilled water to form the PCR master mix. In some embodiments, the PCR master mix has a final volume of about 5 μL, about 6 μL, about 7 μL, about 8 μL, about 9 μL, about 0 μL, about 11 μL, about 12 μL, about 13 μL, about 14 μL, about 15 μL, about 16 μL, about 17 μL, about 18 μL, about 19 μL or about 20 μL or more. In some embodiments, it was found that 2 μL of a genomic DNA sample prepared as described above, are combined with only about 0.15 μL of a 40× real-time PCR assay mix and 2.85 μL of distilled water in order to form the PCR master mix.

While exemplary reactions are described herein, one of skill would understand how to modify the temperatures and times based on the probe design. Moreover, the present methods contemplate any combination of the above times and temperatures.

In some embodiments, primers are tested and designed in a laboratory setting. In some embodiments, primers are designed by computer based in silico methods. Primer sequences are based on the sequence of the amplicon or target nucleic acid sequence that is to be amplified. Shorter amplicons typically replicate more efficiently and lead to more efficient amplification as compared to longer amplicons.

In designing primers, one of skill would understand the need to take into account melting temperature (Tₘ; the temperature at which half of the primer-target duplex is dissociated and becomes single stranded and is an indication of duplex stability; increased Tₘ indicates increased stability) based on GC and AT content of the primers being designed as well as secondary structure considerations (increased GC content can lead to increased secondary structure). Tₘ's can be calculated using a variety of methods known in the art and those of skill would readily understand such various methods for calculating Tₘ; such methods include for example but are not limited to those available in online tools such as the Tₘ calculators available on the World Wide Web at promega.com/techserv/tools/biomath/calc11.htm. Primer specificity is defined by its complete sequence in combination with the 3′ end sequence, which is the portion elongated by Taq polymerase. In some embodiments, the 3′ end should have at least 5 to 7 unique nucleotides not found anywhere else in the target sequence, in order to help reduce false-priming and creation of incorrect amplification products. Forward and reverse primers typically bind with similar efficiency to the target. In some instances, tools such as NCBI BLAST (located on the World Wide Web at ncbi.nlm.nih.gov) are employed to performed alignments and assist in primer design.

Those of skill in the art would be well aware of the basics regarding primer design for a target nucleic acid sequence and a variety of reference manuals and texts have extensive teachings on such methods, including for example, Molecular Cloning (three volume set, Cold Spring Harbor Laboratory Press, 2012) and Current Protocols (Genetics and Genomics; Molecular Biology; 2003-2013) and Real-Time PCR in Microbiology: From Diagnostics to Characterization (Ian M. MacKay, Calster Academic Press; 2007); PrimerAnalyser Java tool available on the World Wide Web at primerdigital.com/tools/PrimerAnalyser.html and Kalendar R, et al. (Genomics, 98(2): 137-144 (2011)), all of which are incorporated herein in their entireties for all purposes.

An additional aspect of primer design is primer complexity or linguistic sequence complexity (see, Kalendar R, et al. (Genomics, 98(2): 137-144 (2011)). Primers with greater linguistic sequence complexity (e.g., nucleotide arrangement and composition) are typically more efficient. In some embodiments, the linguistic sequence complexity calculation method is used to search for conserved regions between compared sequences for the detection of low-complexity regions including simple sequence repeats, imperfect direct or inverted repeats, polypurine and polypyrimidine triple-stranded cDNA structures, and four-stranded structures (such as G-quadruplexes). In some embodiments, linguistic complexity (LC) measurements are performed using the alphabet-capacity L-gram method (see, A. Gabrielian, A. Bolshoy, Computer & Chemistry 23:263-274 (1999) and Y. L. Orlov, V. N. Potapov, Complexity: an internet resource for analysis of DNA sequence complexity, Nucleic Acids Res. 32: W628-W633(2004)) along the whole sequence length and calculated as the sum of the observed range (xi) from 1 to L size words in the sequence divided by the sum of the expected (E) value for this sequence length. Some G-rich (and C-rich) nucleic acid sequences fold into four-stranded DNA structures that contain stacks of G-quartets (see, the World Wide Web at quadruplex.org). In some instances, these quadruplexes are formed by the intermolecular association of two or four DNA molecules, dimerization of sequences that contain two G-bases, or by the intermolecular folding of a single strand containing four blocks of guanines (see, P. S. Ho, PNAS, 91:9549-9553 (1994); I. A. Il'icheva, V. L. Florent'ev, Russian Journal of Molecular Biology 26:512-531(1992); D. Sen, W. Gilbert, Methods Enzymol. 211:191-199 (1992); P. A. Rachwal, K. R. Fox, Methods 43:291-301 (2007); S. Burge, G. N. Parkinson, P. Hazel, A. K. Todd, K. Neidle, Nucleic Acids Res. 34:5402-5415 (2006); A. Guédin, J. Gros, P. Alberti, J. Mergny, Nucleic Acids Res. 38:7858-7868 (2010); O. Stegle, L. Payet, J. L. Mergny, D. J. MacKay, J. H. Leon, Bioinformatics 25:1374-i382 (2009); in some instances, these are eliminated from primer design because of their low linguistic complexity, LC=32% for (TTAGGG)₄.

These methods include various bioinformatics tools for pattern analysis in sequences having GC skew, (G−C)/(G+C), AT skew, (A−T)/(A+T), CG-AT skew, (S−W)/(S+W), or purine-pyrimidine (R−Y)/(R+Y) skew regarding CG content and melting temperature and provide tools for determining linguistic sequence complexity profiles. For example the GC skew in a sliding window of n, where n is a positive integer, bases is calculated with a step of one base, according to the formula, (G−C)/(G+C), in which G is the total number of guanines and C is the total number of cytosines for all sequences in the windows (Y. Benita, et al., Nucleic Acids Res. 31:e99 (2003)). Positive GC-skew values indicated an overabundance of G bases, whereas negative GC-skew values represented an overabundance of C bases. Similarly, other skews are calculated in the sequence. Such methods, as well as others, are employed to determine primer complexity in some embodiments.

According to non-limiting example embodiments, real-time PCR is performed using exonuclease primers (TAQMAN® probes). In such embodiments, the primers utilize the 5′ exonuclease activity of thermostable polymerases such as Taq to cleave dual-labeled probes present in the amplification reaction (See, e.g., Wittwer, C. et al. Biotechniques 22:130-138, 1997). While complementary to the PCR product, the primer probes used in this assay are distinct from the PCR primer and are dually-labeled with both a molecule capable of fluorescence and a molecule capable of quenching fluorescence. When the probes are intact, intramolecular quenching of the fluorescent signal within the DNA probe leads to little signal. When the fluorescent molecule is liberated by the exonuclease activity of Taq during amplification, the quenching is greatly reduced leading to increased fluorescent signal. Non-limiting examples of fluorescent probes include the 6-carboxy-fluorescein moiety and the like. Exemplary quenchers include Black Hole Quencher 1 moiety and the like.

A variety of PCR primers can find use with the disclosed methods. Exemplary primers include but are not limited to those described herein.

A variety of detection probes can find use with the disclosed methods and are employed for genotyping and or for quantification. Detection probes commonly employed by those of skill in the art include but are not limited to hydrolysis probes (also known as TAQMAN® probes, 5′ nuclease probes or dual-labeled probes), hybridization probes, and Scorpion primers (which combine primer and detection probe in one molecule).

In some embodiments, detection probes contain various modifications. In some embodiments, detection probes include modified nucleic acid residues, such as but not limited to 2′-O-methyl ribonucleotide modifications, phosphorothioate backbone modifications, phosphorodithioate backbone modifications, phosphoramidate backbone modifications, methylphosphonate backbone modifications, 3′ terminal phosphate modifications and/or 3′ alkyl substitutions.

In some embodiments, the detection probe has increased affinity for a target sequence due to modifications. Such detection probes include detection probes with increased length, as well as detection probes containing chemical modifications. Such modifications include but are not limited to 2′-fluoro (2′-deoxy-2′-fluoro-nucleosides) modifications, LNAs (locked nucleic acids), PNAs (peptide nucleic acids), ZNAs (zip nucleic acids), morpholinos, methylphosphonates, phosphoramidates, polycationic conjugates and 2′-pyrene modifications. In some embodiments, the detector probes contains one or more modifications including 2′ fluoro modifications (aka, 2′-Deoxy-2′-fluoro-nucleosides), LNAs (locked nucleic acids), PNAs (peptide nucleic acids), ZNAs (zip nucleic acids), morpholinos, methylphosphonates, phosphoramidates, and/or polycationic conjugates.

In some embodiments, the detection probes contain detectable moieties, such as those described herein as well as any detectable moicties known to those of skill in the art. Such detectable moieties include for example but are not limited to fluorescent labels and chemiluminescent labels. Examples of such detectable moieties can also include members of FRET pairs. In some embodiments, the detection probe contains a detectable entity.

Examples of fluorescent labels include but are not limited to AMCA, DEAC (7-Diethylaminocoumarin-3-carboxylic acid); 7-Hydroxy-4-methylcoumarin-3; 7-Hydroxycoumarin-3; MCA (7-Methoxycoumarin-4-acetic acid); 7-Methoxycoumarin-3; AMF (4′-(Aminomethyl)fluorescein); 5-DTAF (5-(4,6-Dichlorotriazinyl)aminofluorescein); 6-DTAF (6-(4,6-Dichlorotriazinyl)aminofluorescein); 6-FAM (6-Carboxyfluorescein; aka FAM; including TAQMAN® FAM™); TAQMAN VIC®; 5(6)-FAM cadaverine; 5-FAM cadaverine; 5(6)-FAM ethylenediamme; 5-FAM ethylenediamme; 5-FITC (FITC Isomer I; fluorescein-5-isothiocyanate); 5-FITC cadaverin; Fluorescein-5-maleimide; 5-IAF (5-lodoacetamidofluorescein); 6-JOE (6-Carboxy-4′,5′-dichloro-2′,7′-dimethoxyfluorescein); 5-CR110 (5-Carboxyrhodamine 110); 6-CR110 (6-Carboxyrhodamine 110); 5-CR6G (5-Carboxyrhodamine 6G); 6-CR6G (6-Carboxyrhodamine 6G); 5(6)-Carboxyrhodamine 6G cadaverine; 5(6)-Carboxyrhodamine 6G ethylenediamme; 5-ROX (5-Carboxy-X-rhodamine); 6-ROX (6-Carboxy-X-rhodamine); 5-TAMRA (5-Carboxytetramethylrhodamine); 6-TAMRA (6-Carboxytetramethylrhodamine); 5-TAMRA cadaverine; 6-TAMRA cadaverine; 5-TAMRA ethylenediamme; 6-TAMRA ethylenediamme; 5-TMR C6 maleimide; 6-TMR C6 maleimide; TR C2 maleimide; TR cadaverine; 5-TRITC; G isomer (Tetramethylrhodamine-5-isothiocyanate); 6-TRITC; R isomer (Tetramethylrhodamine-6-isothiocyanate); Dansyl cadaverine (5-Dimethylaminonaphthalene-1-(N-(5-aminopentyl))sulfonamide); EDANS C2 maleimide; fluorescamine; NBD; and pyrromethene and derivatives thereof.

Examples of chemiluminescent labels include but are not limited to those labels used with Southern Blot and Western Blot protocols (see, for e.g., Sambrook and Russell, Molecular Cloning: A Laboratory Manual, (3rd ed.) (2001); incorporated by reference herein in its entirety). Examples include but are not limited to -(2′-spiroadamantane)-4-methoxy-4-(3″-phosphoryloxy)phenyl-1,2-dioxetane (AMPPD); acridinium esters and adamantyl-stabilized 1,2-dioxetanes, and derivatives thereof.

The labeling of probes is known in the art. The labeled probes are used to hybridize within the amplified region during amplification. The probes are modified so as to avoid them from acting as primers for amplification. The detection probe is labeled with two fluorescent dyes, one capable of quenching the fluorescence of the other dye. One dye is attached to the 5′ terminus of the probe and the other is attached to an internal site, so that quenching occurs when the probe is in a non-hybridized state.

Typically, real-time PCR probes consist of a pair of dyes (a reporter dye and an acceptor dye) that are involved in fluorescence resonance energy transfer (FRET), whereby the acceptor dye quenches the emission of the reporter dye. In general, the fluorescence-labeled probes increase the specificity of amplicon quantification.

Real-time PCR that are used in some embodiments of the disclosed methods also include the use of one or more hybridization probes (i.e., detection probes), as determined by those skilled in the art, in view of this disclosure. By way of non-limiting example, such hybridization probes include but are not limited to one or more of those provided in the described methods. Exemplary probes, such as the HEX channel and/or FAM channel probes, are understood by one skilled in the art.

According to example embodiments, detection probes and primers are conveniently selected e.g., using an in silico analysis using primer design software and cross-referencing against the available nucleotide database of genes and genomes deposited at the National Center for Biotechnology Information (NCBI). Some additional guidelines may be used for selection of primers and/or probes in some embodiments. For example, in some embodiments, the primers and probes are selected such that they are close together, but not overlapping. In some embodiments, the primers may have the same (or close T^{M}) (e.g., between about 58° C. and about 60° C.). In some embodiments, the T_{M} of the probe is approximately 10° C. higher than that selected for the T_{M} of the primers. In some embodiments, the length of the probes and primers is selected to be between about 17 and 39 base pairs, etc. These and other guidelines are used in some instances by those skilled in the art in selecting appropriate primers and/or probes.

In some embodiments, the SNP described herein may be detected by melting curve analysis using the detection probes above. For example, the melting curves of short oligonucleotide probes hybridized to a region containing the SNP of interest may be analyzed. Two probes are used in these reactions, each one being complimentary to a particular allele at the SNP in question. Perfectly matched probes are more stable and have a higher melting temperature compared to mismatched probes. Hence, SNP genotypes are inferred according to the characteristic melting curves produced by annealing and melting either matched or mismatched oligonucleotide probes.

In one aspect, the methods described herein may include detecting the ten or more genetic variations or SNPs described herein by hybridizing at least one detection probe to a nucleotide molecule from a sample or its amplicons and detecting the at least one detection probe.

In another aspect, diagnostic testing is employed to determine one or more genetic conditions by detection of any of a variety of mutations. In some embodiments, diagnostic testing is used to confirm a diagnosis when a particular condition is suspected based on for example physical manifestations, signs and/or symptoms as well as family history information. In some embodiments, the results of a diagnostic test assist those of skill in the medical arts in determining an appropriate treatment regimen for a given subject and allow for more personalized and more effective treatment regimens. In some embodiments, a treatment regimen includes any of a variety of pharmaceutical treatments, surgical treatments, lifestyles changes or a combination thereof as determined by one of skill in the art.

The nucleic acids obtained by the disclosed methods are useful in a variety of diagnostic tests, including tests for detecting mutations such as deletions, insertions, transversions and transitions. In some embodiments, such diagnostics are useful for identifying unaffected individuals who carry one copy of a gene for a disease that requires two copies for the disease to be expressed, identifying unaffected individuals who carry one copy of a gene for a disease in which the information could find use in developing a treatment regimen, preimplantation genetic diagnosis, prenatal diagnostic testing, newborn screening, genealogical DNA test (for genetic genealogy purposes), presymptomatic testing for predicting or diagnosing KC.

In some embodiments, newborns can be screened. In some embodiments, newborn screening includes any genetic screening employed just after birth in order to identify genetic disorders. In some embodiments, newborn screening finds use in the identification of genetic disorders so that a treatment regimen is determined early in life. Such tests include but are not limited to testing infants for phenylketonuria and congenital hypothyroidism.

In some embodiments, carrier testing is employed to identify people who carry a single copy of a gene mutation. In some cases, when present in two copies, the mutation can cause a genetic disorder. In some cases, one copy is sufficient to cause a genetic disorder. In some cases, the presence of two copies is contra-indicated for a particular treatment regimen, such as the presence of the Avellino mutation and pre-screening prior to performing surgical procedures in order to ensure the appropriate treatment regimen is pursued for a given subject. In some embodiments, such information is also useful for individual contemplating procreation and assists individuals with making informed decisions as well as assisting those skilled in the medical arts in providing important advice to individual subjects as well as subjects' relatives.

In some embodiments, predictive and/or presymptomatic types of testing are used to detect gene mutations associated with a variety of disorders. In some cases, these tests are helpful to people who have a family member with a genetic disorder, but who may exhibit no features of the disorder at the time of testing. In some embodiments, predictive testing identifies mutations that increase a person's chances of developing disorders with a genetic basis, including for example but not limited to certain types of cancer. In some embodiments, presymptomatic testing is useful in determining whether a person will develop a genetic disorder, before any physical signs or symptoms appear. The results of predictive and presymptomatic testing provides information about a person's risk of developing a specific disorder and help with making decisions about an appropriate medical treatment regimen for a subject as well as for a subject's relatives. Predictive testing is also employed, in some embodiments, to detect mutations which are contra-indicated with certain treatment regimens, such as the presence of the Avellino mutation being contra-indicated with performing LASIK surgery and/or other refractive procedures, such as but not limited to Phototherapeutic keratectomy (PTK) and/or Photorefractive keratectomy (PRK). For example, subjects exhibiting the Avellino mutation should not undergo LASIK surgery or other refractive procedures. Similarly, in some cases, subjects with KC mutation(s) should not undergo LASIK surgery or other refractive procedures.

In some embodiments, diagnostic testing also includes pharmacogenomics which includes genetic testing that determines the influence of genetic variation on drug response. Information from such pharmacogenomic analyses finds use in determining and developing an appropriate treatment regimen. Those of skill in the medical arts employ information regarding the presence and/or absence of a genetic variation in designing appropriate treatment regimen.

In some embodiments, diseases whose genetic profiles are determined using the methods of the present disclosure include KC.

In some embodiments, the present methods find use in development of personalized medicine treatment regimens by providing the genomic DNA which is used in determining the genetic profile for an individual. In some embodiments, such genetic profile information is employed by those skilled in the art in order determine and/or develop a treatment regimen. In some embodiments, the presence and/or absence of various genetic variations and mutations identified in nucleic acids isolated by the described methods are used by those of skill in the art as part of a personalized medicine treatment regimen or plan. For example, in some embodiments, information obtained using the disclosed methods is compared to databases or other established information in order to determine a diagnosis for a specified disease and or determine a treatment regimen. In some cases, the information regarding the presence or absence of a genetic mutation in a particular subject is compared to a database or other standard source of information in order to make a determination regarding a proposed treatment regimen. In some cases, the presence of a genetic mutation indicates pursuing a particular treatment regimen. In some cases the absence of a genetic mutation indicates not pursuing a particular treatment regimen.

In some embodiments, information regarding the presence and/or absence of a particular genetic mutation is used to determine the treatment efficacy of treatment with the therapeutic entity, as well as to tailor treatment regimens for treatment with therapeutic entity. In some embodiments, information regarding the presence and/or absence of a genetic mutation is employed to determine whether to pursue a treatment regimen. In some embodiments, information regarding the presence and/or absence of a genetic mutation is employed to determine whether to continue a treatment regimen. In some embodiments, the presence and/or absence of a genetic mutation is employed to determine whether to discontinue a treatment regimen. In other embodiments, the presence and/or absence of a genetic mutation is employed to determine whether to modify a treatment regimen. In some embodiments the presence and/or absence of a genetic mutation is used to determine whether to increase or decrease the dosage of a treatment that is being administered as part of a treatment regimen. In other embodiments, the presence and/or absence of a genetic mutation is used to determine whether to change the dosing frequency of a treatment administered as part of a treatment regimen. In some embodiments, the presence and/or absence of a genetic mutation is used to determine whether to change the number of dosages per day, per week, times per day of a treatment. In some embodiments the presence and/or absence of a genetic mutation is used to determine whether to change the dosage amount of a treatment. In some embodiments, the presence and/or absence of a genetic mutation is determined prior to initiating a treatment regimen and/or after a treatment regimen has begun. In some embodiments, the presence and/or absence of a genetic mutation is determined and compared to predetermined standard information regarding the presence or absence of a genetic mutation.

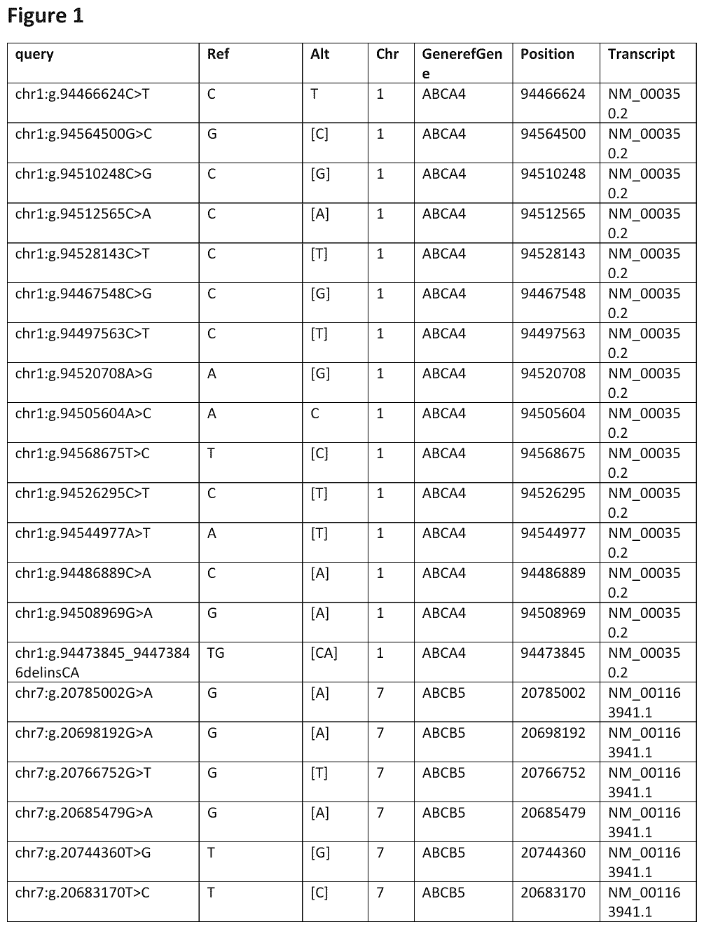

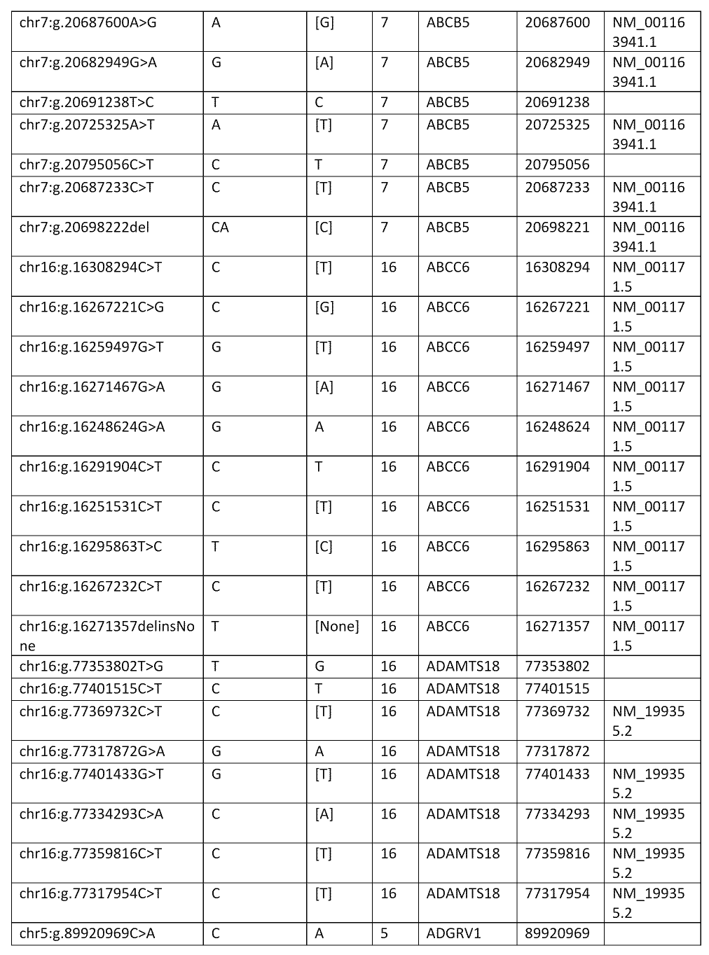

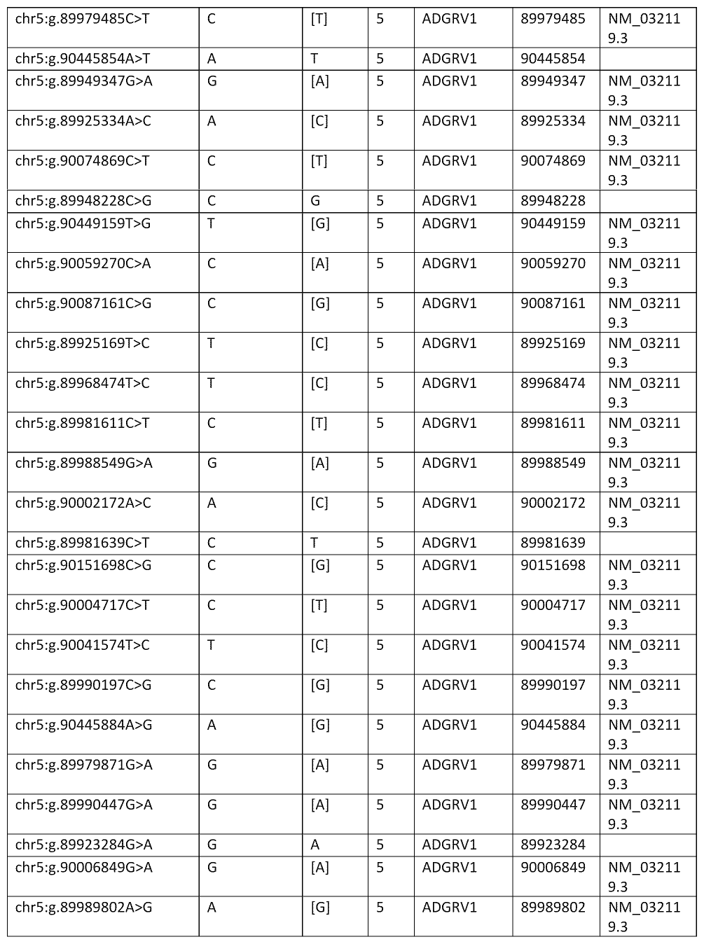

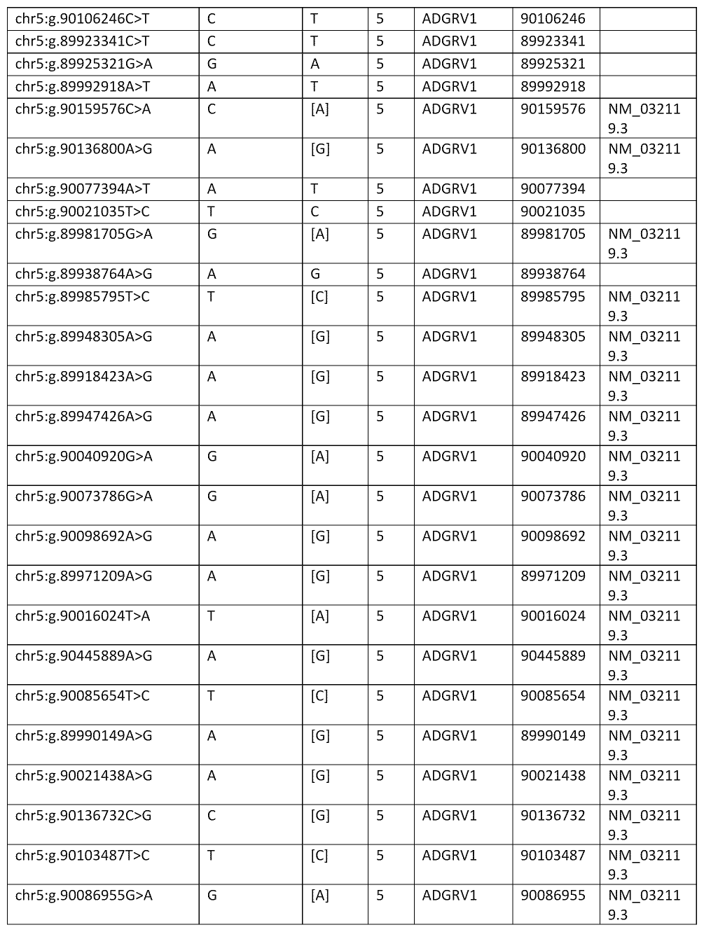

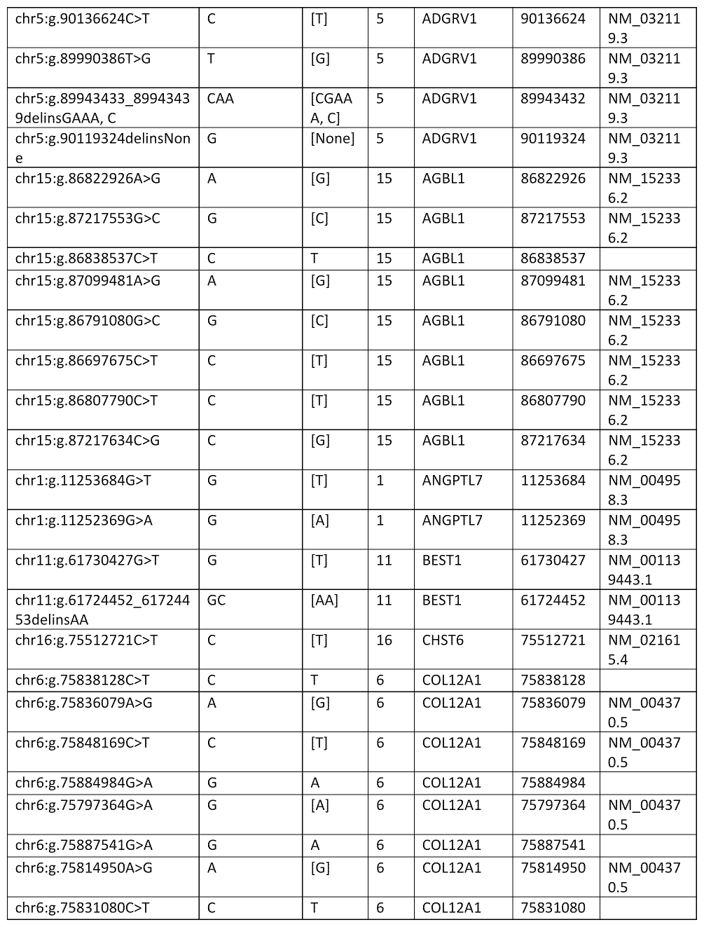

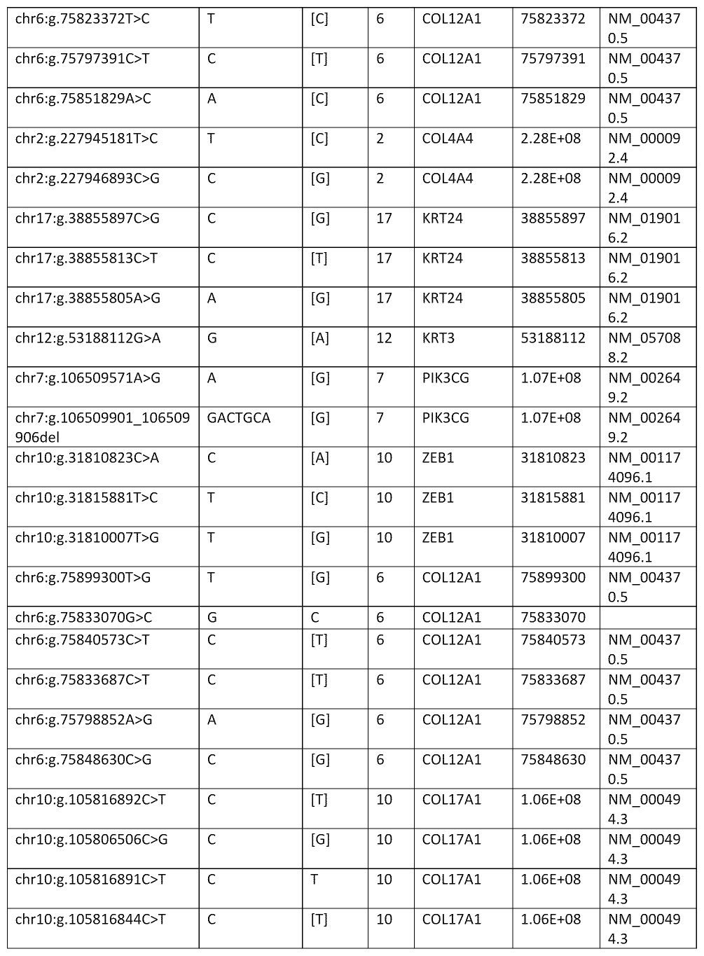

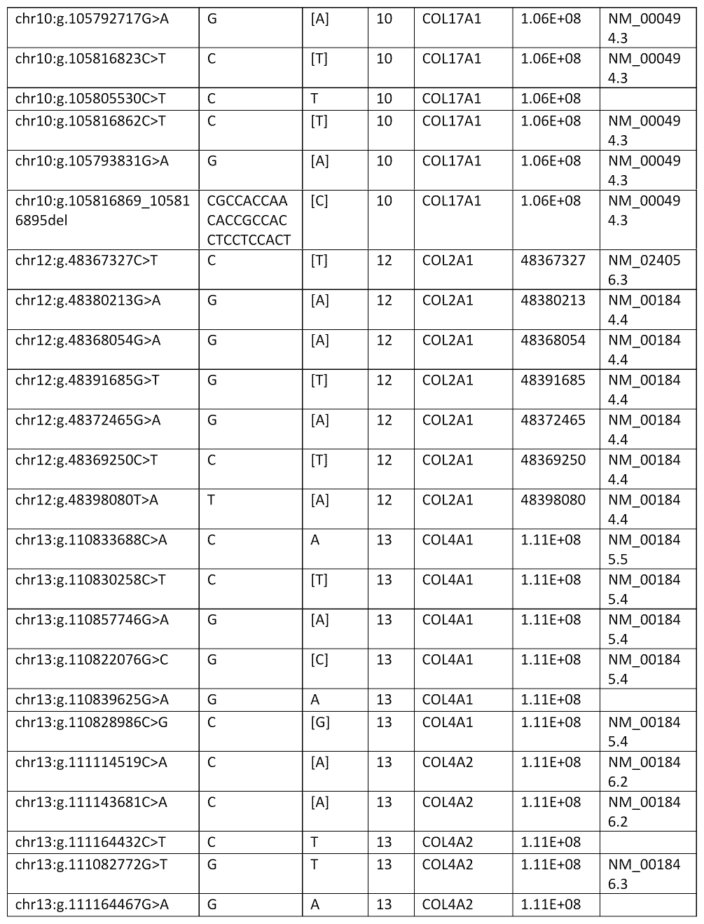

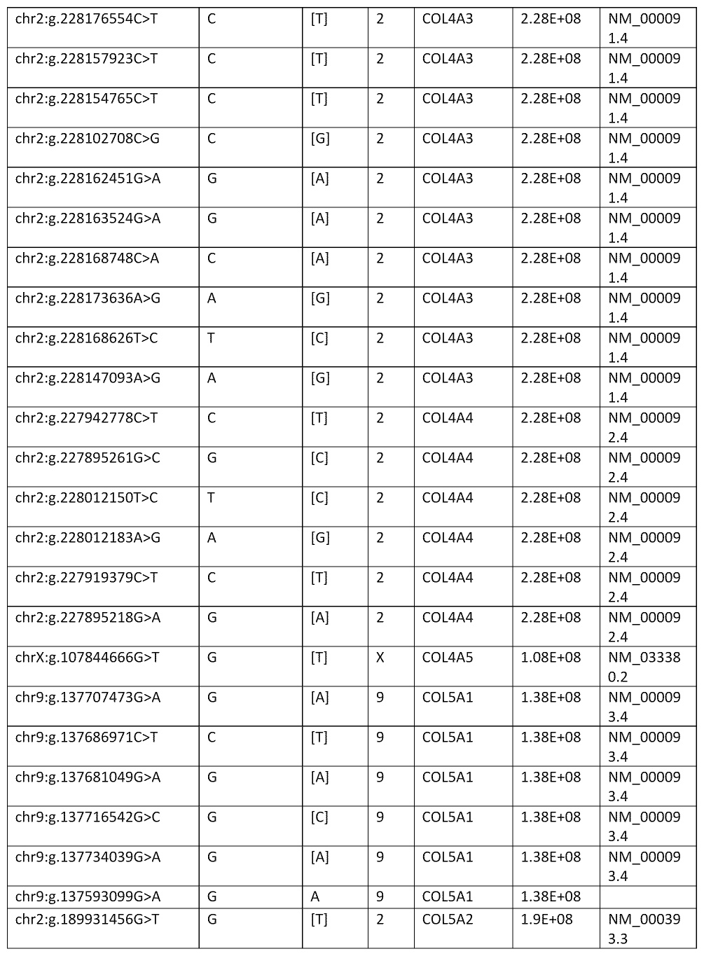

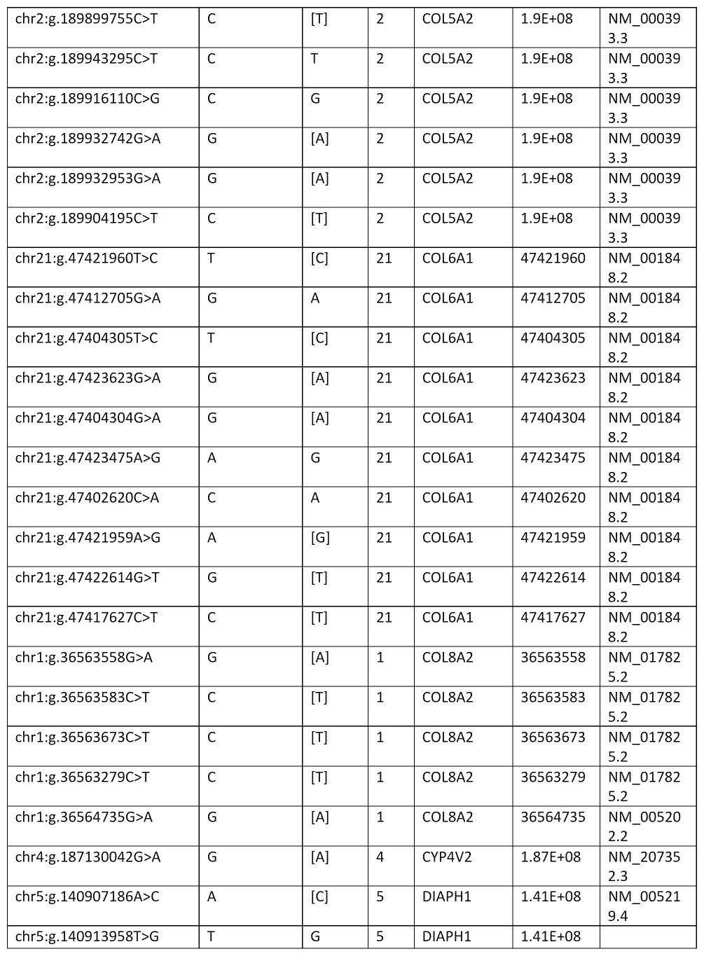

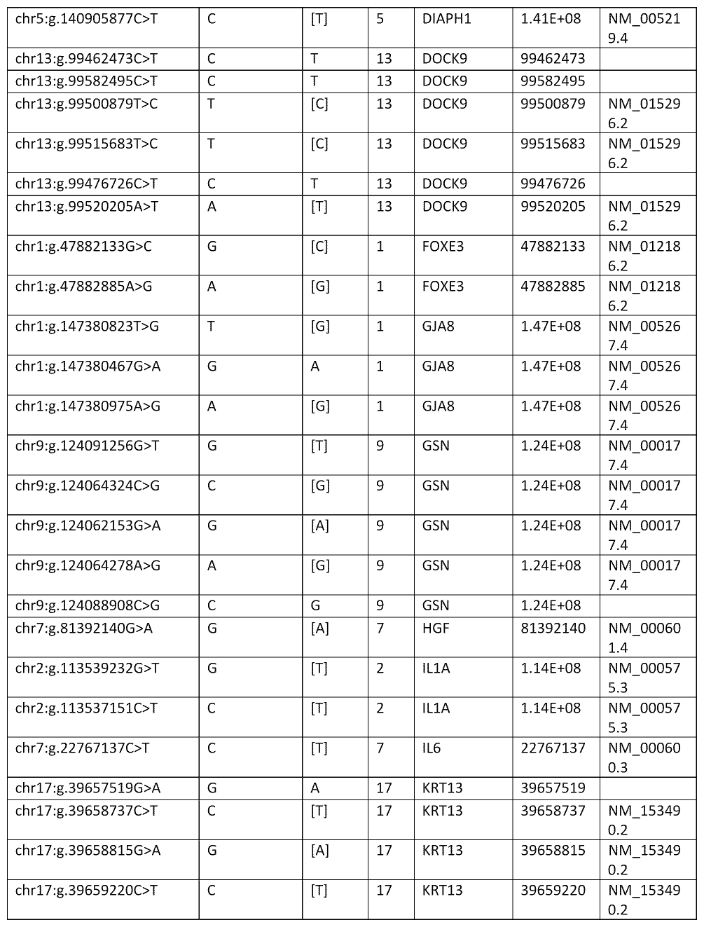

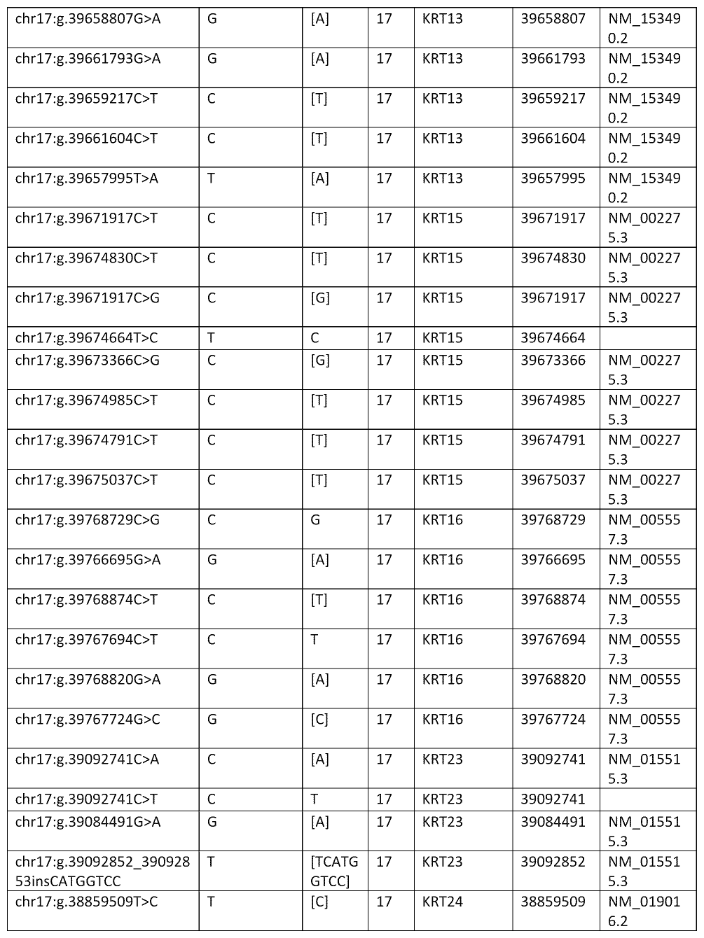

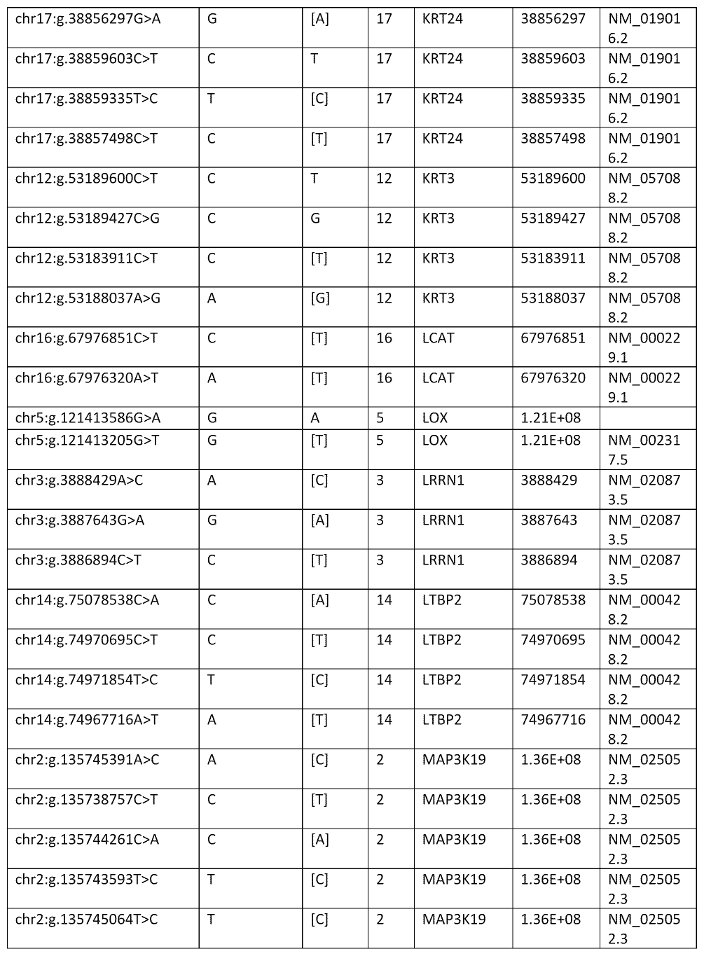

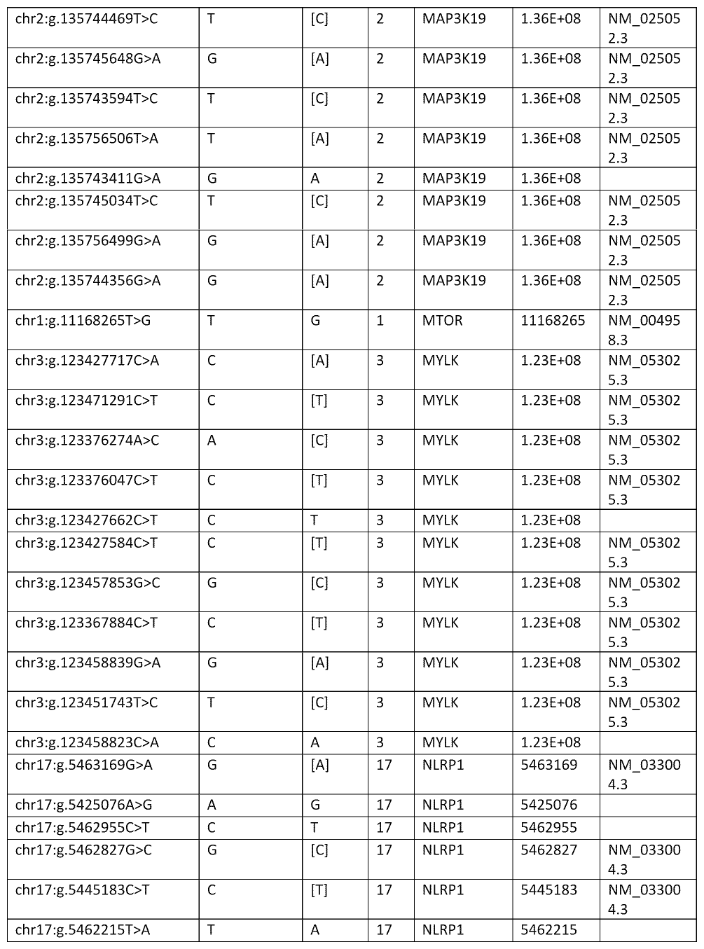

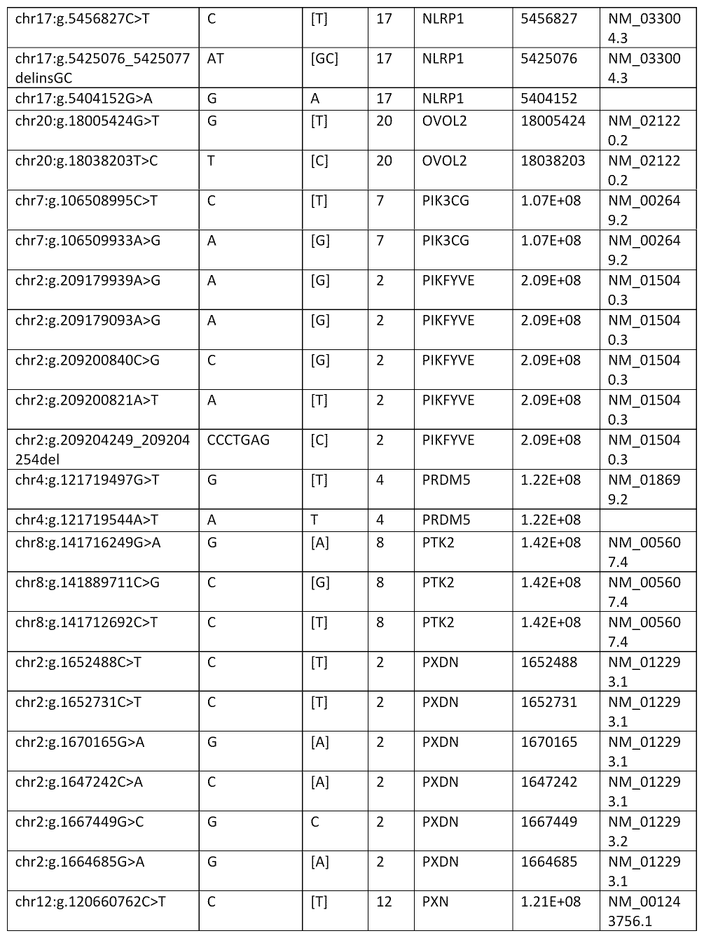

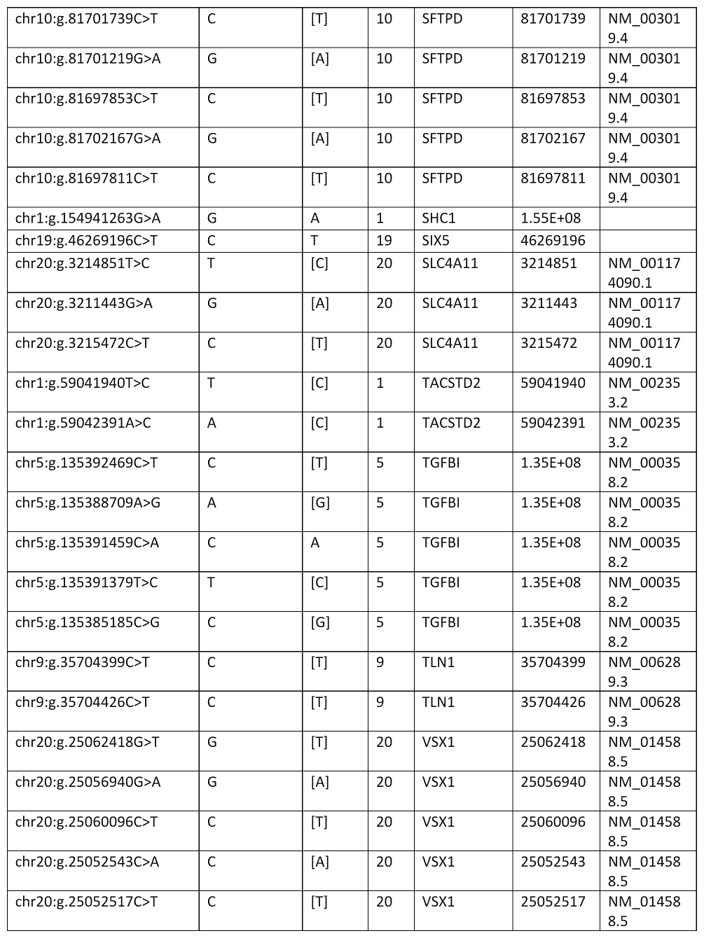

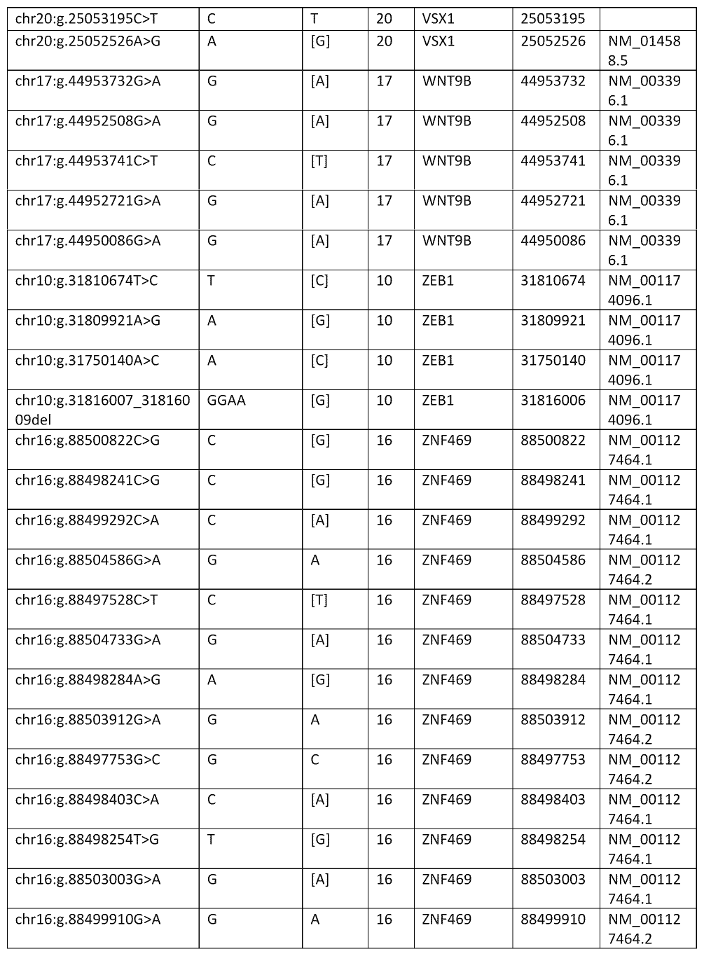

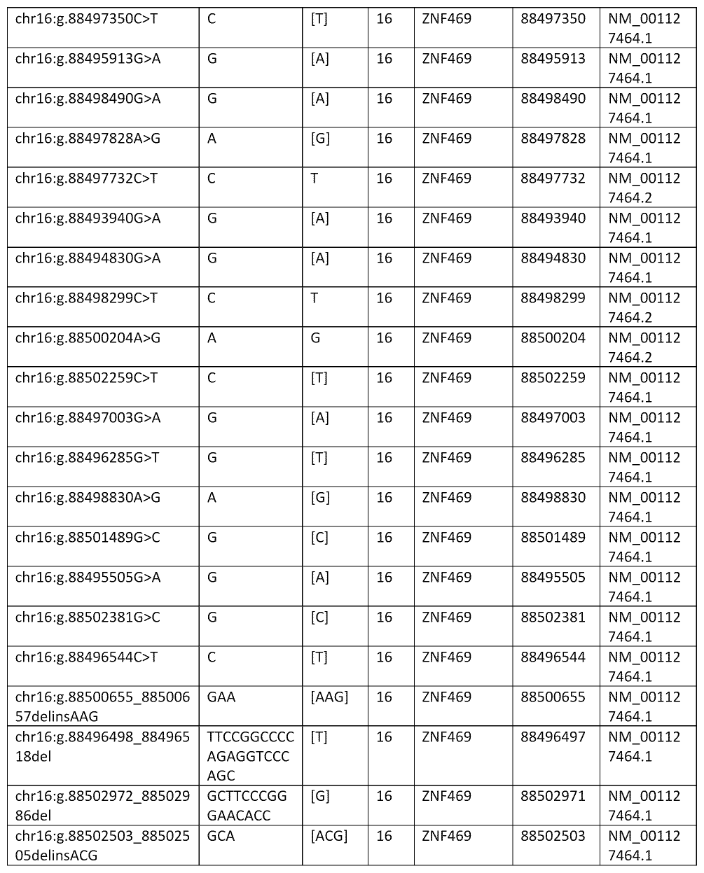

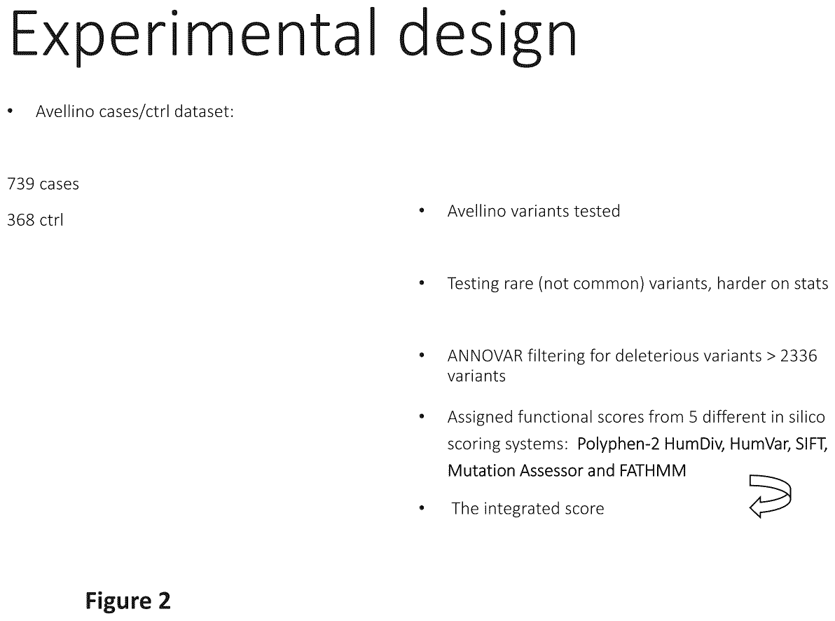

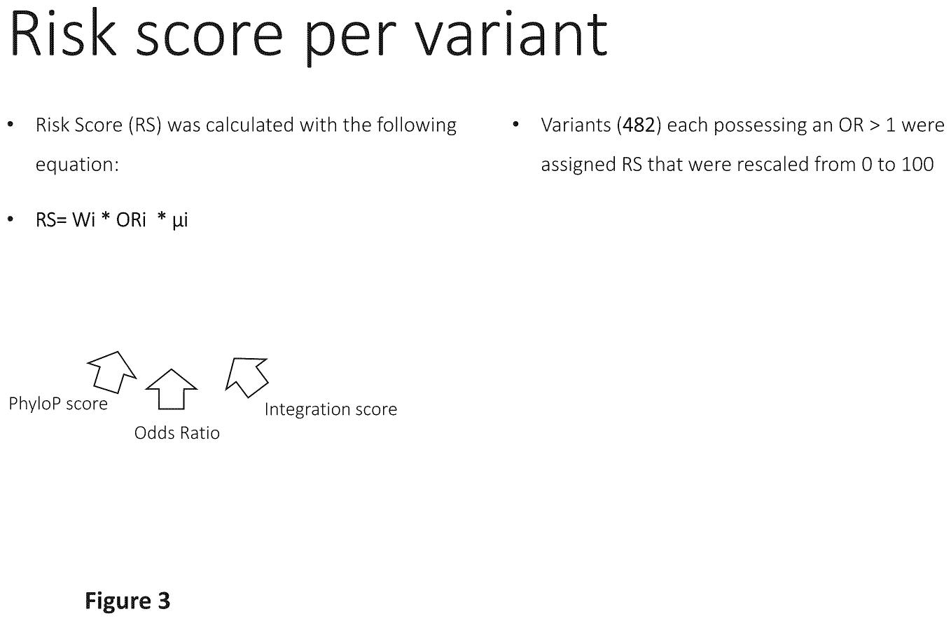

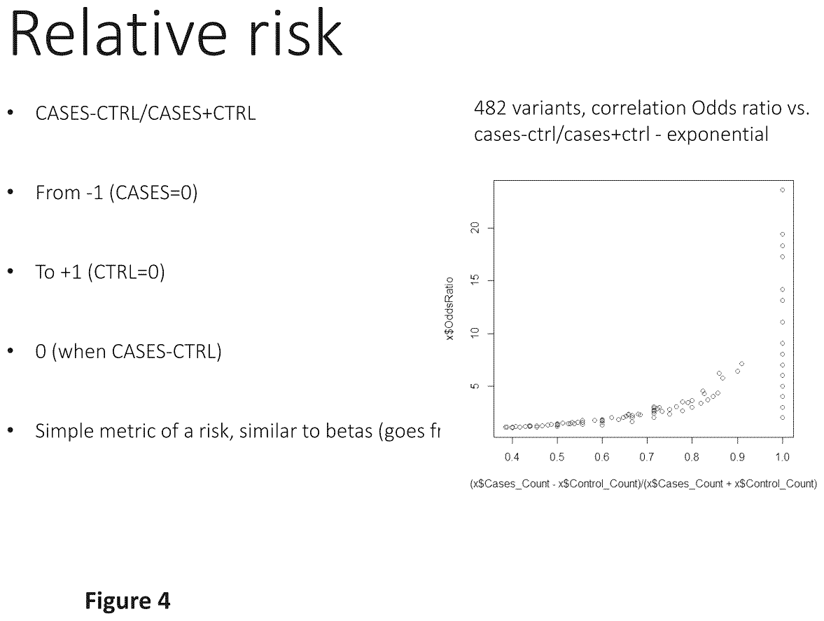

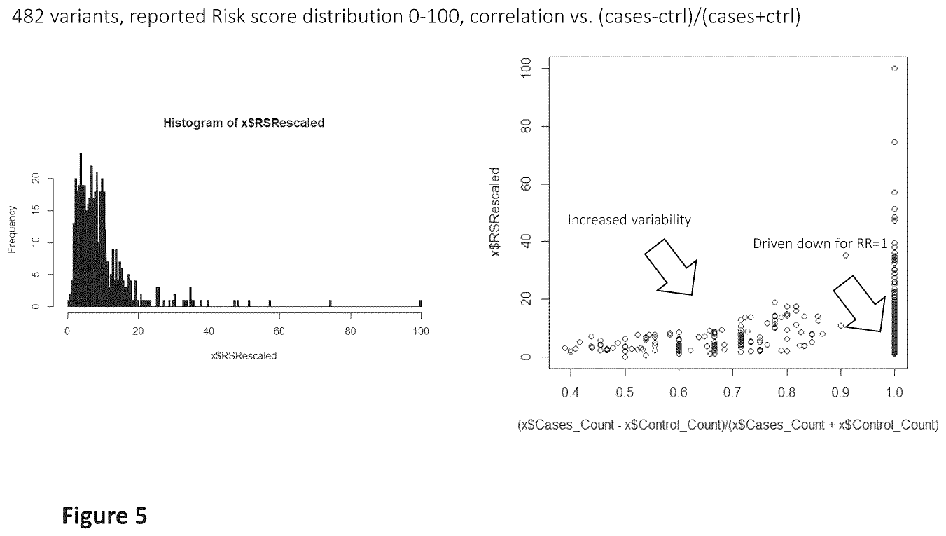

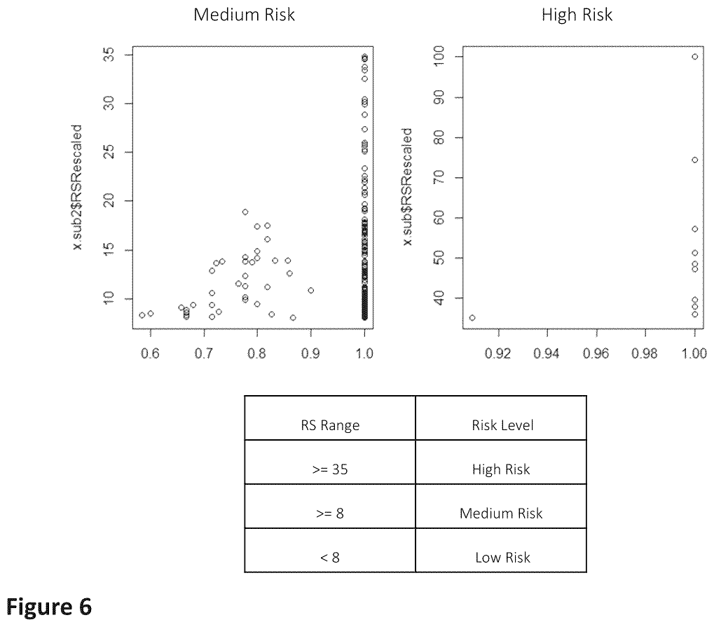

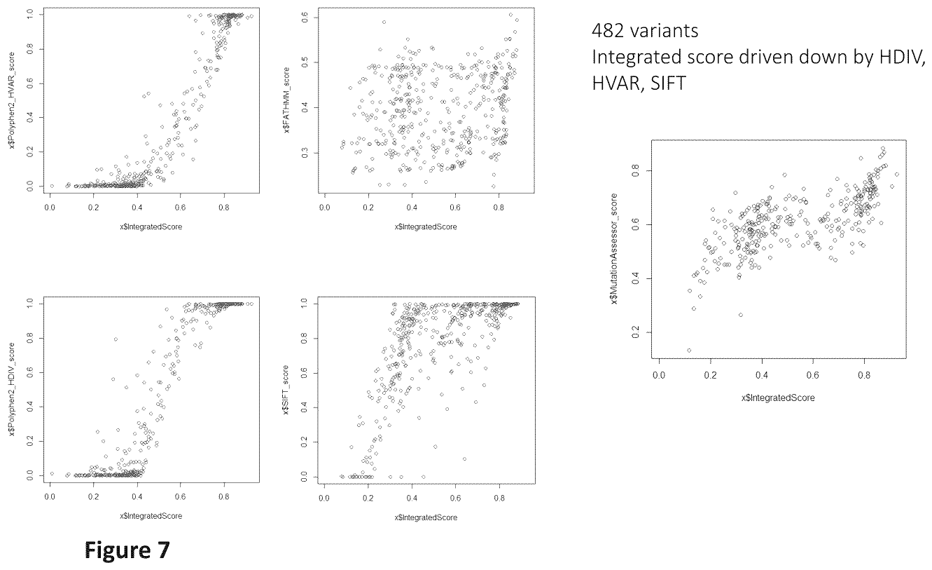

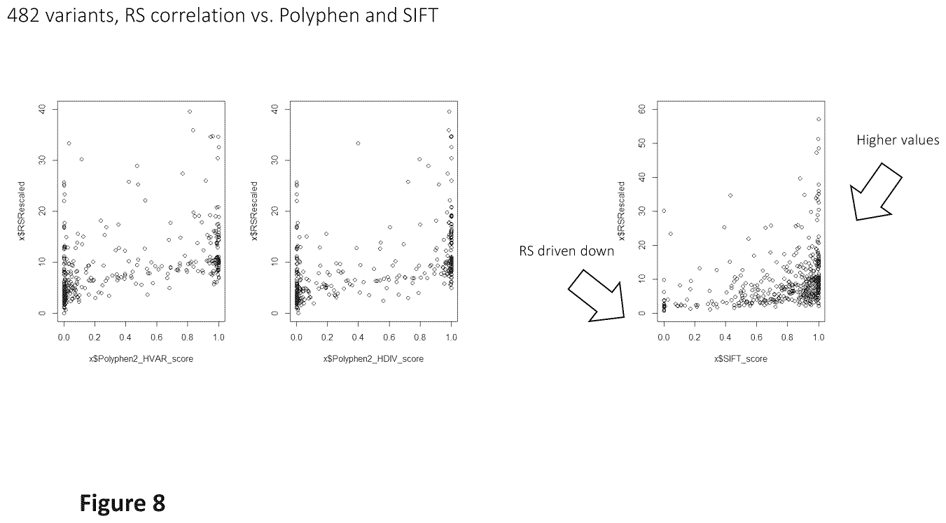

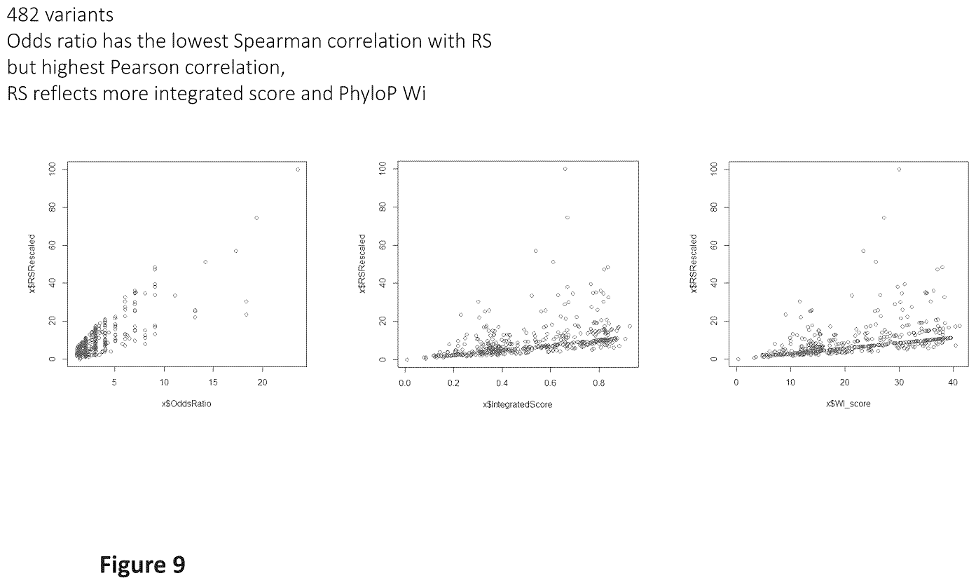

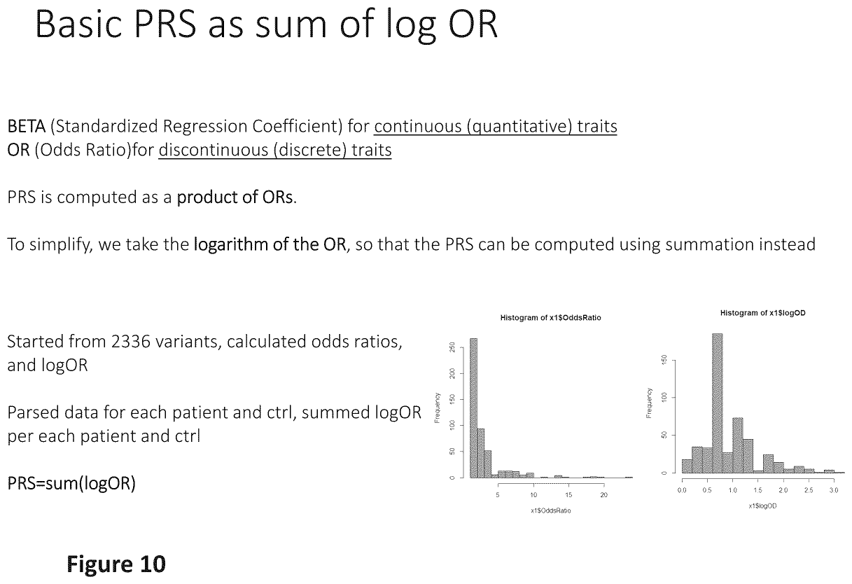

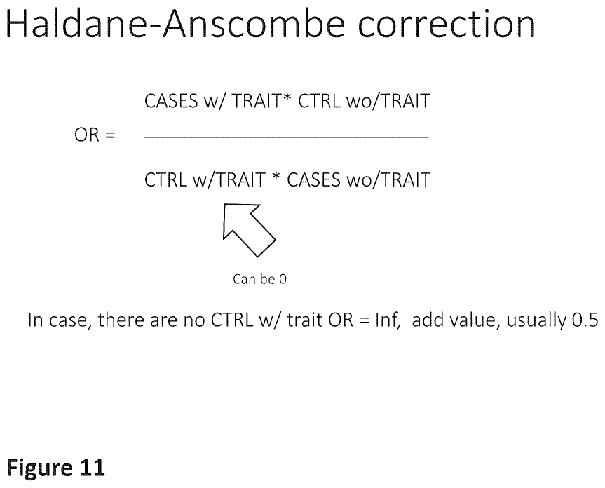

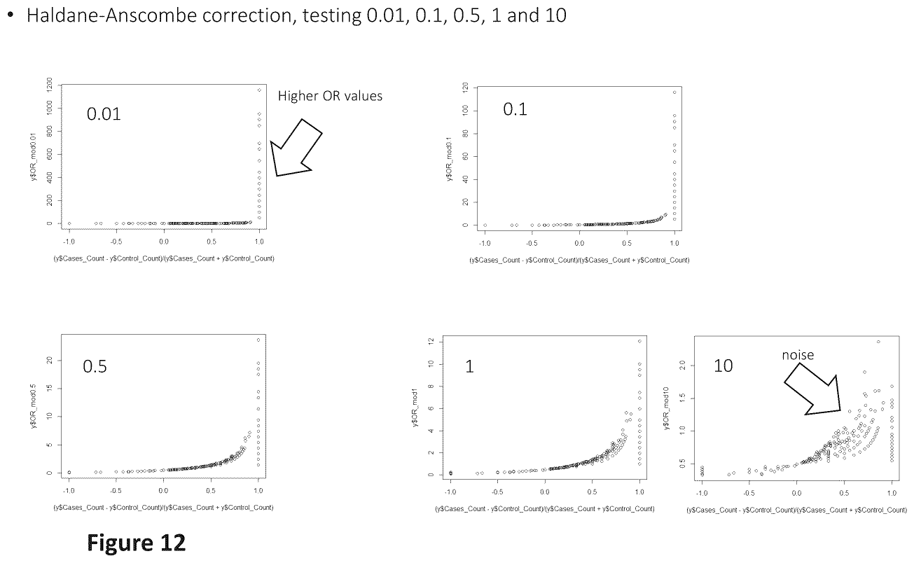

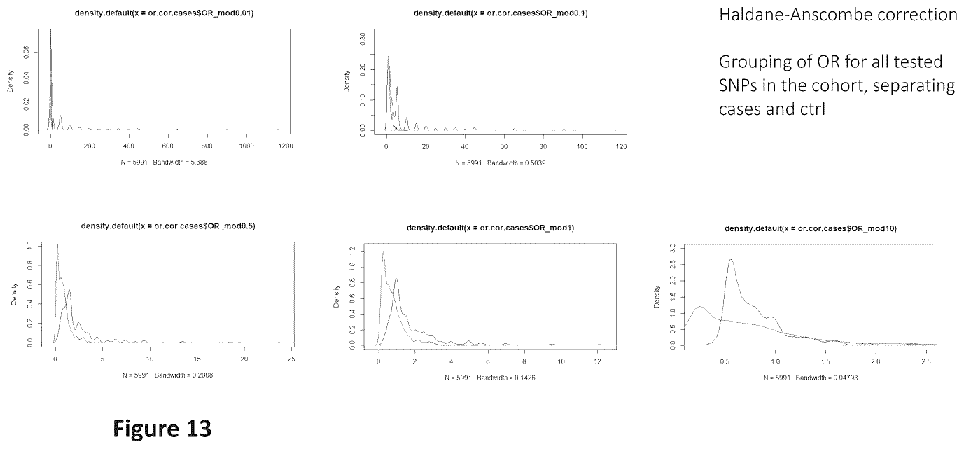

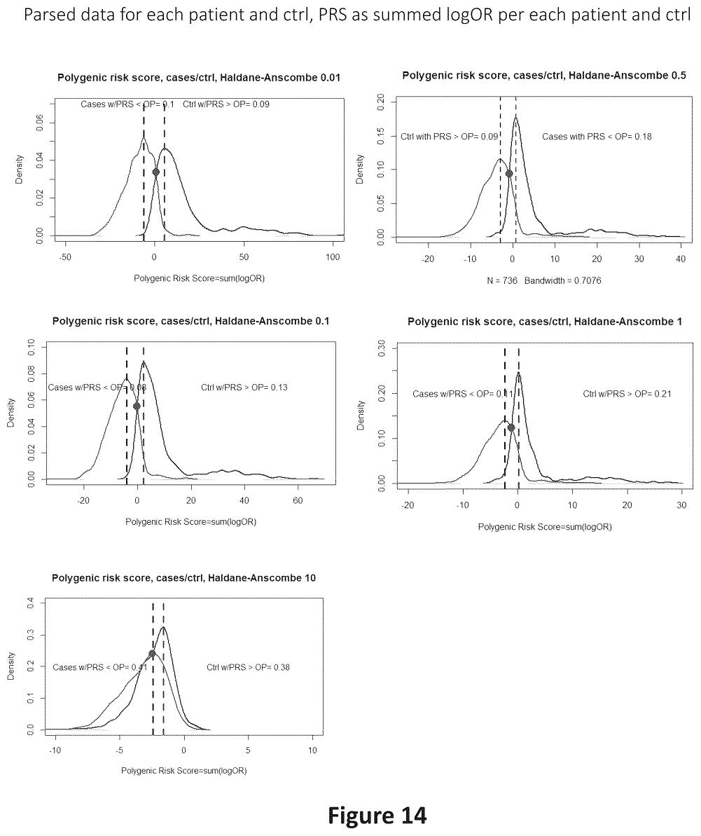

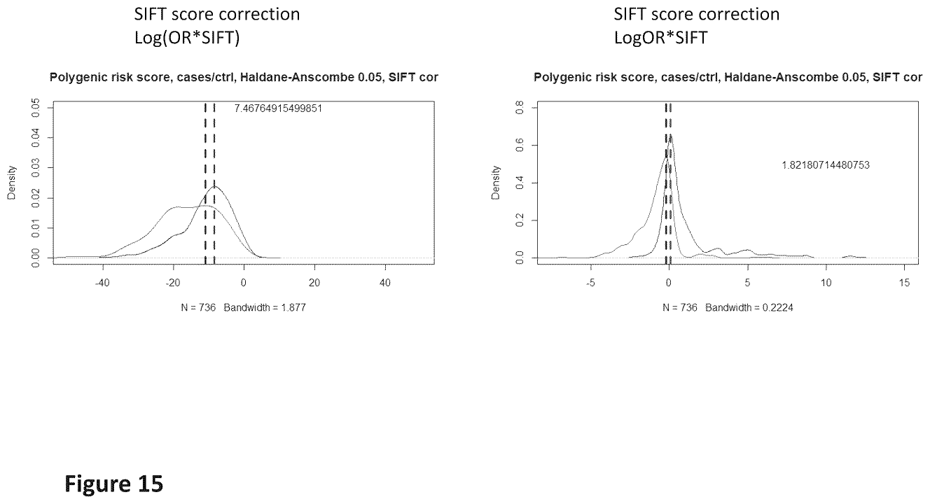

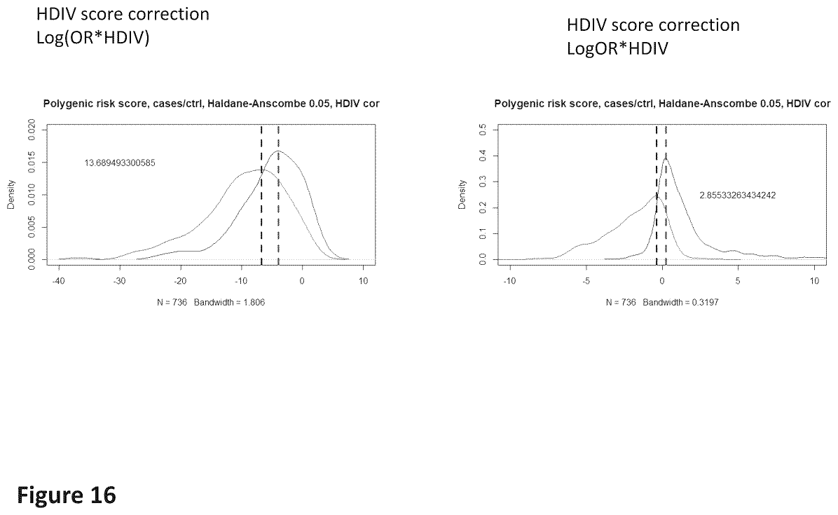

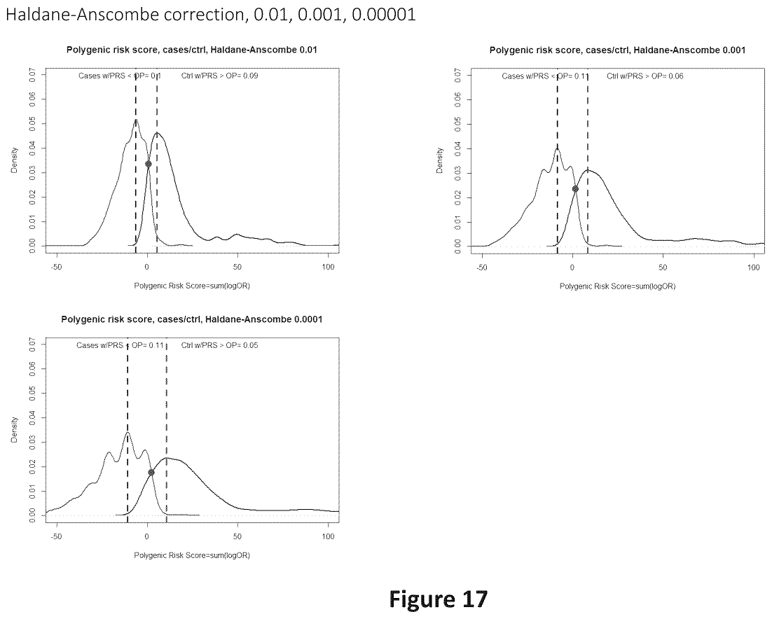

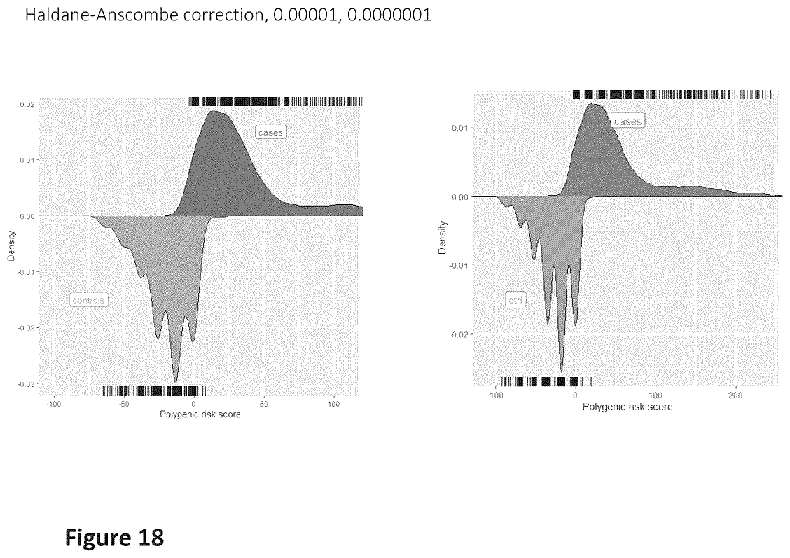

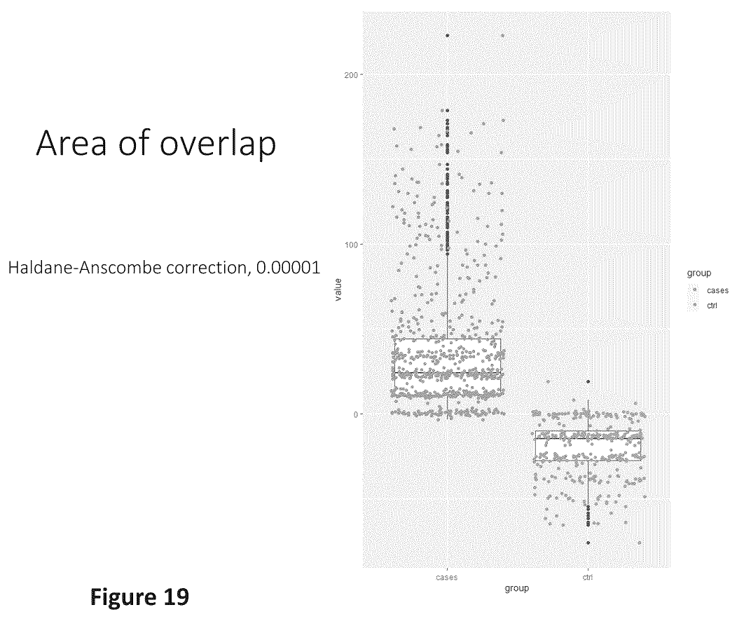

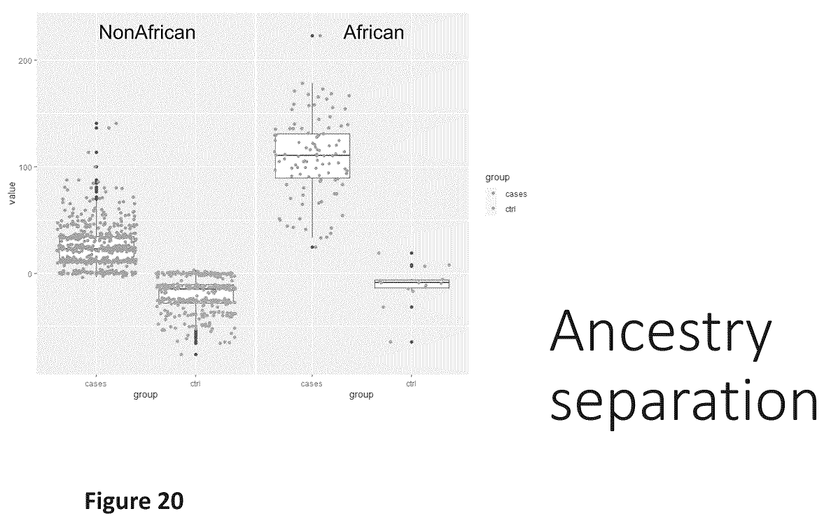

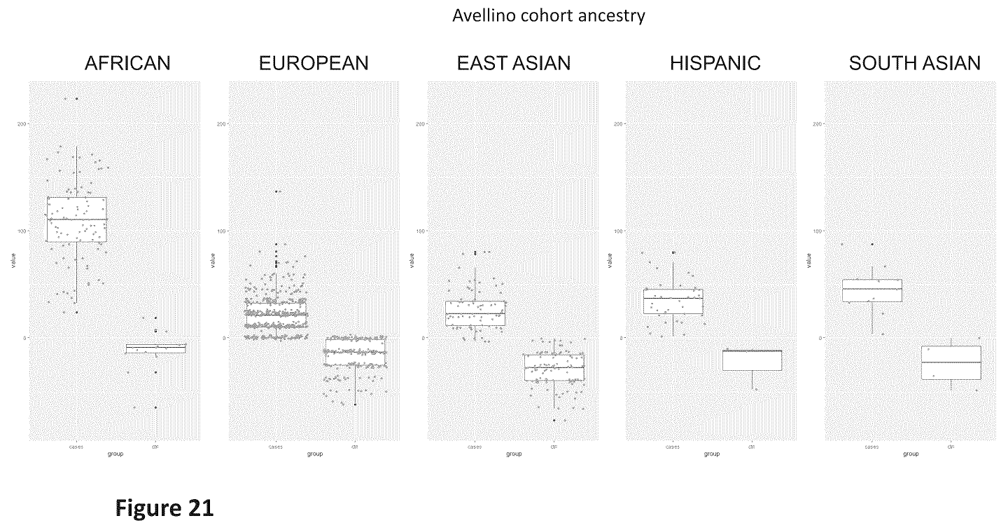

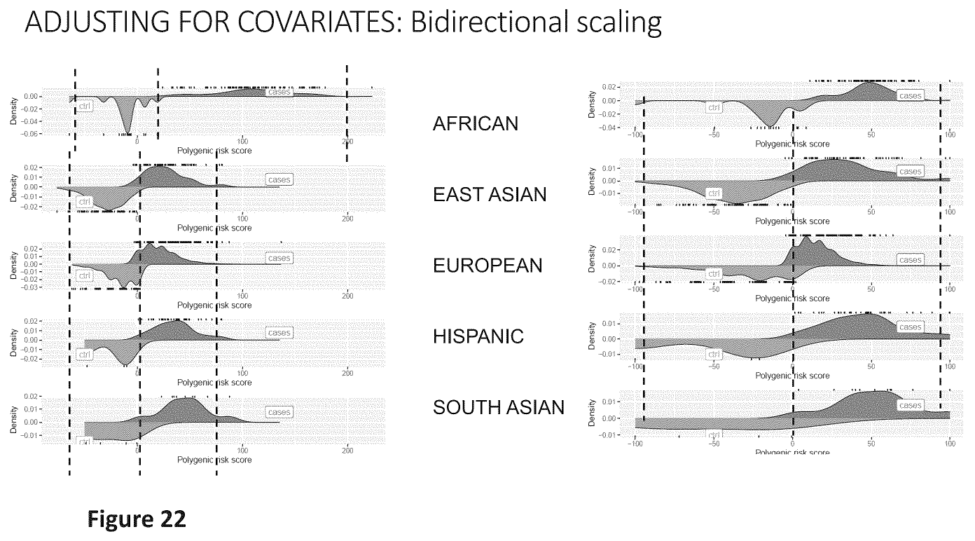

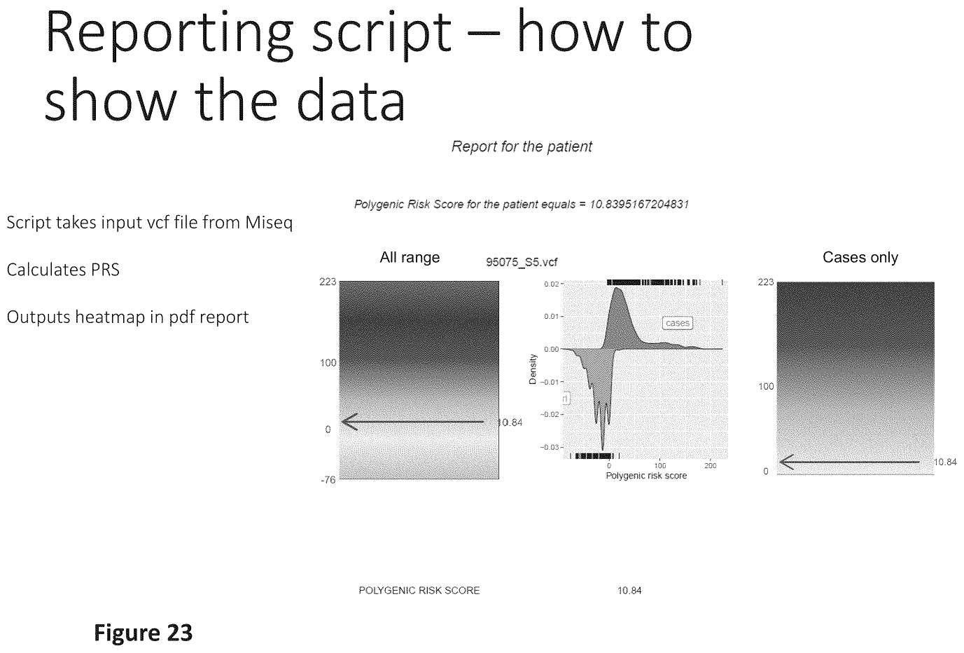

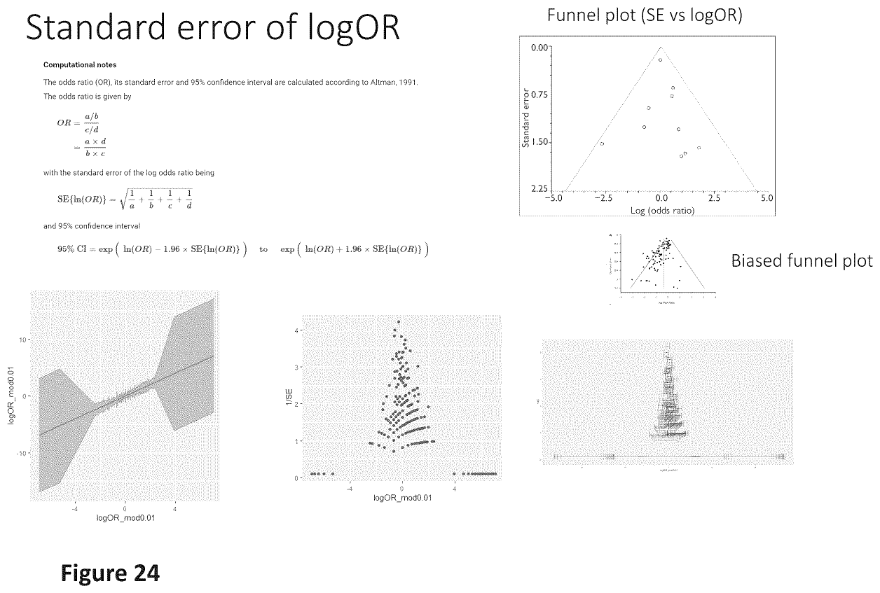

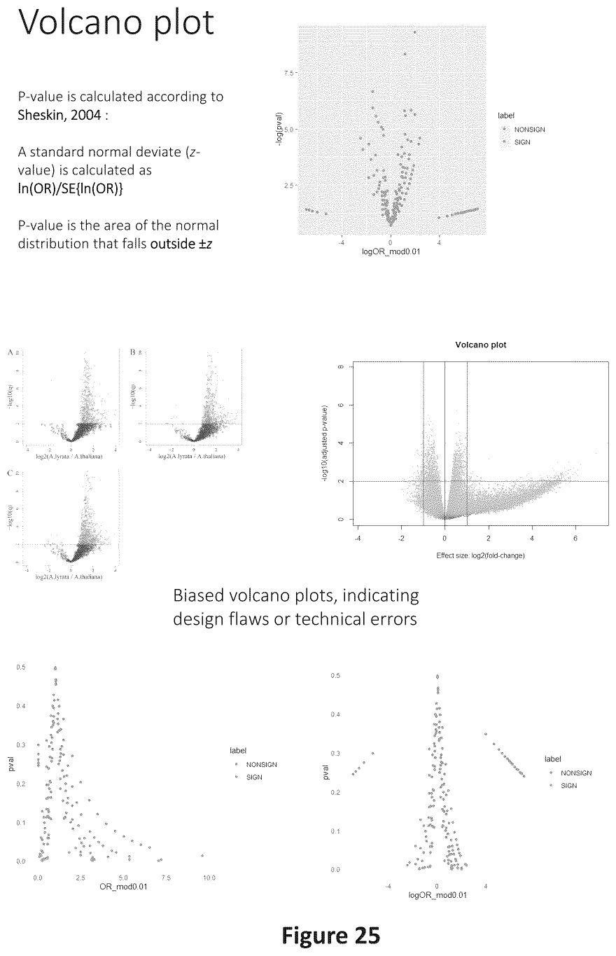

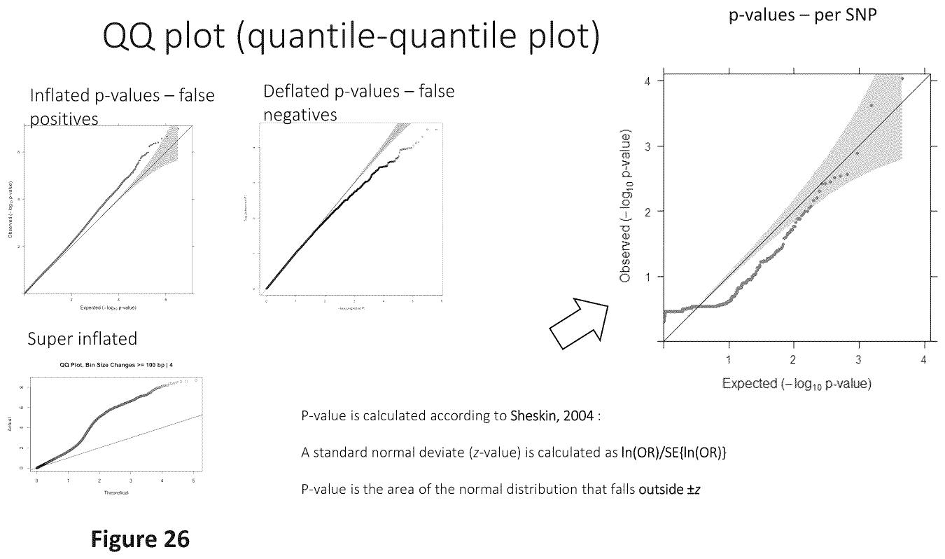

In some embodiments, a composite of the presence and/or absence of more than one genetic mutation is generated using the disclosed methods and such composite includes any collection of information regarding the presence and/or absence of more than one genetic mutation. In some embodiments, the presence or absence of genetic mutations in ten or more genes is examined and used for generation of a composite. For example, genetic variants from 10, 11, 12, 13, 14, 15, 16, 17, 18, 19, 20, 21, 22, 23, 24, 25, 30, 35, 40, 45, 46, 47, 48, 49, 50, 51, 52, 53, 54, 55 or more genes selected from the group consisting of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469 is examined and used for generation of a composite. In additional embodiments, the genetic variants may be selected from ten or more genes selected from the group consisting of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469. In some embodiments, at least one genetic variant in each of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469 is examined and used for generation of a composite. In some embodiments, the presence or absence of at least 10, 11, 12, 13, 14, 15, 16, 17, 18, 19, 20, 21, 22, 23, 24, 25, 30, 35, 40, 45, 46, 47, 48, 49, 50, 51, 52, 53, 54, 55, 60, 70, 80, 90, 100, 150, 200, 250, 300, 350, 400, 411 or 482 genetic mutations, for example, including those in FIG. 1, is examined and used for generation of a composite. Exemplary information in some embodiments includes nucleic acid or protein information, or a combination of information regarding both nucleic acid and/or protein genetic mutations. Generally, the composite includes information regarding the presence and/or absence of a genetic mutation. In some embodiments, these composites are used for comparison with predetermined standard information in order to pursue, maintain or discontinue a treatment regimen.

In some embodiments, KC is predicted and/or detected for example through detection of genetic variants of ten or more genes as described herein. In some embodiments, KC is predicted and/or detected for example through detection of ten or more genetic variants from different genes, for example, including at least 10, 11, 12, 13, 14, 15, 16, 17, 18, 19, 20, 21, 22, 23, 24, 25, 30, 35, 40, 45, 46, 47, 48, 49, 50, 51, 52, 53, 54, 55 genes selected from but not limited to the group consisting of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469. In some embodiments, the genetic variants are selected from at least 10, 11, 12, 13, 14, 15, 16, 17, 18, 19, 20, 21, 22, 23, 24, 25, 30, 35, 40, 45, 46, 47, 48, 49, 50, 51, 52, 53, 54, 55 genes selected from the group consisting of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469. In some embodiments, the variants include at least one variant selected from the group consisting of ABCA4, ABCB5, ABCC6, ADAMTS18, ADGRV1, ANGPTL7, BEST1, COL2A1, COL4A1, COL4A2, COL4A3, COL4A4, COL5A2, COL6A1, COL12A1, DIAPH1, DOCK9, FYN, HGF, ILIA, ILIRN, IL6, IL10, ITGB1, KRT13, KRT15, KRT16, KRT23, KRT24, LOX, LRRN1, LTBP2, MAP2K1, MAP3K19, MTOR, MYLK, NLRP1, PAX6, PIK3CG, PIK3R1, PTK2, PXDN, PXN, RAF1, RHOA, SFTPD, SHC1, SIX5, TLN1, WNT9A, and WNT9B. In some embodiments, KC is predicted and/or detected for example through detection of at least one genetic variant from each of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469. In some embodiments, the presence or absence of at least 10, 11, 12, 13, 14, 15, 16, 17, 18, 19, 20, 21, 22, 23, 24, 25, 30, 35, 40, 45, 46, 47, 48, 49, 50, 51, 52, 53, 54, 55, 60, 70, 80, 90, 100, 150, 200, 250, 300, 350, 400, 411 or 482 genetic mutations, for example, including those in FIGS. 1, are detected.

The present disclosure also provides methods to assist with differential diagnosis. In some embodiments, KC is distinguished from pellucid marginal degeneration, keratoglobus, contact lens induced corneal warpage, and/or corneal ectasia post excimer laser treatment through detection of genetic variants of the ten or more genes described herein, for example, including at least 10, 11, 12, 13, 14, 15, 16, 17, 18, 19, 20, 21, 22, 23, 24, 25, 30, 35, 40, 45, 46, 47, 48, 49, 50, 51, 52, 53, 54, 55 genes selected from but not limited to the group consisting of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469. In some embodiments, at least one genetic variant from the group consisting of ABCA4, ABCB5, ABCC6, ADAMTS18, ADGRV1, ANGPTL7, BEST1, COL2A1, COL4A1, COL4A2, COL4A3, COL4A4, COL5A2, COL6A1, COL12A1, DIAPH1, DOCK9, FYN, HGF, ILIA, ILIRN, IL6, IL10, ITGB1, KRT13, KRT15, KRT16, KRT23, KRT24, LOX, LRRN1, LTBP2, MAP2K1, MAP3K19, MTOR, MYLK, NLRP1, PAX6, PIK3CG, PIK3R1, PTK2, PXDN, PXN, RAF1, RHOA, SFTPD, SHC1, SIX5, TLN1, WNT9A, and WNT9B is detected. In some embodiments, at least one genetic variant from each of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469 is detected. In some embodiments, the presence or absence of at least 10, 11, 12, 13, 14, 15, 16, 17, 18, 19, 20, 21, 22, 23, 24, 25, 30, 35, 40, 45, 46, 47, 48, 49, 50, 51, 52, 53, 54, 55, 60, 70, 80, 90, 100, 150, 200, 250, 300, 350, 400, 411 or 482 genetic mutations, for example, including those in FIGS. 1, are detected.

In some embodiments, the genetic variants are selected from the group consisting of genetic variants listed in FIG. 1. The genetic variants described herein, for example, include at least 10, 11, 12, 13, 14, 15, 16, 17, 18, 19, 20, 21, 22, 23, 24, 25, 30, 35, 40, 45, 46, 47, 48, 49, 50, 51, 52, 53, 54, 55, 60, 70, 80, 90, 100, 150, 200, 250, 300, 350, 400, 411 or 482 variants selected from but not limited to those listed in FIG. 1.

In some embodiments, the subject is Afro-American.

In some embodiments, the subject is Caucasian.

In some embodiments, the subject is Hispanic.

In some embodiments, the subject is East Asian.

In some embodiments, the detection of the genetic variants described herein is combined with a physical examination in order to diagnose KC or predict the risk of developing KC. Such a physical examination can include an eye examination as well as ancillary tests to assess corneal curvature, astigmatism and thickness. In some embodiments, the best potential vision of the subject is evaluated. Components of the eye exam can include but are not limited to medical history (including, for example, change in eye glass prescription, decreased vision, history of eye rubbing, medical problems, allergies, and/or sleep patterns); assessment of relevant aspects of the subject's mental and physical status; visual acuity with current correction (the power of the present correction recorded) at distance and when appropriate at near and far distances; measurement of best corrected visual acuity with spectacles and/or hard or gas permeable contact lenses (with refraction when indicated); measurement of pinhole visual acuity; external examination (lids, lashes, lacrimal apparatus, orbit); examination of ocular alignment and motility; assessment of pupillary function; measurement of intraocular pressure (IOP); slit-lamp biomicroscopy of the anterior segment; dilated examination (including for example, dilated examination of the lens, macula, peripheral retina, optic nerve, and vitreous); and Keratometry/Computerized Topography/Computerized Tomography/Ultrasound Pachymetry.

In some embodiments, the detection of the genetic variants described herein is in combination with one or more indications or signs of KC development in order to diagnose KC or predict the risk of developing KC. In some embodiments, the sign is an early signs of KC. In some embodiments, an early sign of KC includes but is not limited to asymmetric refractive error with high or progressive astigmatism; keratometry showing high astigmatism and irregularity (axis that do not add to 180 degrees); scissoring of the red reflex on ophthalmoscopy or retinoscopy; inferior steepening, skewed axis, or elevated keratometry values on K reading and computerized corneal topography; corneal thinning, especially in inferior cornea (maximum corneal thinning corresponds to the site of maximum steepening or prominence); Rizzuti's sign or a conical reflection on nasal cornea when a penlight is shone from the temporal side; Fleischer ring, an iron deposit often present within the epithelium around the base of the cone. It is brown in color and best visualized with a cobalt blue filter; or Vogt's striae, fine, roughly vertically parallel striations in the stroma (these generally disappear with firm pressure applied over the eyeball and re-appear when pressure is discontinued). In some embodiments, the sign is a late sign of KC. In some embodiments, a late sign of KC includes but is not limited to Munson's sign (a protrusion of the lower eyelid in downgaze); superficial scarring; break's in Bowman's membrane; acute hydrops (a condition where a break in Descemet's membrane allows aqueous fluid into the stoma causing severe corneal thickening, decreased vision and pain); or stromal scarring after resolution of acute hydrops (which paradoxically may improve vision in some cases by changing corneal curvature and reducing the irregular astigmatism).

In some embodiments, the detection of the genetic variants associated with an increased risk of developing KC described herein can be used to assist with determining a treatment regimen for an individual suspected to have KC or predicted to develop KC in the future.

KC treatment regimens include a variety of treatment regimens directed to providing visual acuity and maintaining sight. Spectacles or soft toric contact lenses in mild cases can be used. Rigid gas permeable contact lenses are needed in the majority of cases to neutralize the irregular corneal astigmatism. The majority of subjects that can wear hard or gas-permeable contact lenses have a dramatic improvement in their vision. Specialty contact lenses have been developed to better fit the irregular and steep corneas found in KC; these include (but not limited to) RoseK™, custom designed contact lenses (based on topography and/or wavefront measurements), semi-scleral contact lenses, piggy back lens use (soft and hard lens used at the same time), and scleral lenses. Subjects that become contact lens intolerant or do not have acceptable vision (e.g., from central scaring) proceed to surgical alternatives.

In some embodiments, the detection of the genetic variants as described herein can be used to begin an appropriate treatment early in an individual suspected to be a risk of developing KC. In some embodiments, treatments are directed to halting changes in the corneal shape. In some embodiments, the detection of the genetic variants that predict and increased risk of developing KC can allow for earlier and/or more frequent monitoring of the cornea in order to identify disease onset at an early stage. (i.e., identify early disease onset).

In some embodiments, treatment includes medical therapy for subjects who have an episode of corneal hydrops involves acute management of the pain and swelling. Subjects are usually given a cycloplegic agent, sodium chloride (Muro) 5% ointment and may be offered a pressure patch. After the pressure patch is removed subjects may still need to continue sodium chloride drops or ointment for several weeks to months until the episode of hydrops has resolved. Subjects are advised to avoid vigorous eye rubbing or trauma.

In another aspect, the detection of the genetic variation or SNPs as described herein can be used to begin early or regular monitoring in an individual suspected to be a risk of developing KC. In some embodiments, subjects can be followed on a 6-month to yearly basis to monitor the progression of the corneal-thinning and steepening and the resultant visual changes and to re-evaluate contact lens fit and care. In some embodiments, subjects who have developed hydrops are seen more frequently until the symptoms resolve.

In another aspect, the detection of the genetic variants as described herein can be used to diagnose KC in a subject. In some embodiments, after diagnosis, a treatment regimen includes surgical interventions. While initial treatment regimens focus on less invasive procedures, such as contact lens fitting if the subject does not exhibit corneal scarring. However, as subjects become intolerant or no longer benefit from contact lenses, surgery is the next option. Surgical options can include but are not limited to INTACS (i.e., implants, also known as ICRS or corneal rings), Anterior lamellar keratoplasty, or penetrating keratoplasty. Treatment can also include non-FDA approved treatments, which include but are not limited to the use of UV/riboflavin collagen cross-linking of the cornea to stiffen the cornea and possibly prevent progressive changes in shape and this treatment can be combined with excimer laser treatment, conductive keratoplasty, and/or INTACS. In some embodiments, surgeons can also use phakic intraocular lenses (IOLs) to address high myopia and some of the astigmatism.

In some embodiments, the surgical intervention includes intracorneal ring segments (INTACS; commercially available from Addition Technology), which have also been approved for the treatment of mild to moderate KC in subjects who are contact lens intolerant. In these cases, subjects must have a clear central cornea and a corneal thickness of >450 microns where the segments are inserted, approximately at 7 mm optical zone. An advantage of INTACS is that they require no removal of corneal tissue, no intraocular incision, and leave the central cornea untouched. Most subjects will need spectacles and/or contact lenses post-operatively for best vision, but will have flatter corneas and easier use of lenses after the procedure. In some instances, INTACS can be removed and then other surgical options can be considered.

In some embodiments, the surgical intervention includes Anterior lamellary keratoplasty, which has resurfaced as an option for treating KC. It involves replacement of the central anterior cornea, leaving the subject's endothelium intact. The advantages are that the risk of endothelial graft rejection is eliminated, and there is less risk of traumatic rupture of the globe in the incision, since the endothelium and Descemet's and some stroma are left intact, and faster visual rehabilitation. There are several techniques including, deep anterior lamellar keratoplasty (DALK) and big bubble keratoplasty (BBK) to remove the anterior stroma, while leaving Descemet's layer and endothelium untouched. However, the procedures can be technically challenging requiring conversation to a penetrating keratoplasty, and post-operatively there is the possibility of interface haze leading to a decrease in best corrected visual acuity (BCVA); it is not clear if astigmatism is better treated with anterior vs penetrating keratoplasty. Penetrating keratoplasty has a high success rate and is the standard surgical treatment with a long track record of safety and efficacy. Risks of this procedure include infection and cornea rejection and risk of traumatic rupture at wound margin. Many subjects after penetrating keratoplasty (PK) may still need hard or gas-permeable contact lenses due to residual irregular astigmatism. Any type of refractive procedure is considered a contraindication in keratoconic subjects due to the unpredictability of the outcome and risk of leading to increased and unstable irregular astigmatism.

Additionally, the treatment for keratoconus includes collagen cross-linking and corneal transplant. Collagen cross-linking is a new treatment that uses a special laser and eyedrops to promote “cross-linking” or strengthening of the collagen fibers that make up the cornea. This treatment may flatten or stiffen the cornea, preventing further protrusion. When good vision is no longer possible with other treatments, a corneal transplant may be recommended. In a corneal transplant, the diseased cornea is removed from your eye and is replaced it with a healthy donor cornea.

In one aspect, the disclosure provides methods for treating keratoconus in a subject, the method comprising diagnosing or prognosing KC and treating KC in the subject. In further embodiments, the treating may comprise wearing eye glasses or contact lenses, administering a cycloplegic agent, applying intracorneal ring segments, performing anterior lamellary keratoplasty, and/or performing collagen cross-linking or corneal transplant.

In another aspect, the disclosure provides a diagnostic kit for diagnosing, prognosing and/or treating KC. Any or all of the reagents described above may be packaged into a diagnostic kit. Such kits include any and/or all of the primers, probes, buffers and/or other reagents described herein in any combination. In some embodiments, the kit includes reagents for detection of the genetic variants described herein. In some embodiments, the reagents are for detecting genetic variants from at least 5, 10, 15, 20, 25, 30, 35, 40, 41, 42, 43, 44, 45, 46, 47, 48, 49, 50, 51, 52, 53, 54, 55 or 56 genes selected from the group consisting of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469. In some embodiments, the reagents are for detecting genetic variants from at least ten genes selected from the group consisting of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469. In some embodiments, the reagents are for detecting at least one genetic variant from the group consisting of ABCA4, ABCB5, ABCC6, ADAMTS18, ADGRV1, ANGPTL7, BEST1, COL2A1, COL4A1, COL4A2, COL4A3, COL4A4, COL5A2, COL6A1, COL12A1, DIAPH1, DOCK9, FYN, HGF, ILIA, ILIRN, IL6, IL10, ITGB1, KRT13, KRT15, KRT16, KRT23, KRT24, LOX, LRRN1, LTBP2, MAP2K1, MAP3K19, MTOR, MYLK, NLRP1, PAX6, PIK3CG, PIK3R1, PTK2, PXDN, PXN, RAF1, RHOA, SFTPD, SHC1, SIX5, TLN1, WNT9A, and WNT9B. In some embodiments, the reagents are for detecting at least one genetic variant from each of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469. In some embodiments, the genetic variants are selected from the group consisting of genetic variants listed in FIG. 1. The genetic variants described herein, for example, include at least 10, 11, 12, 13, 14, 15, 16, 17, 18, 19, 20, 21, 22, 23, 24, 25, 30, 35, 40, 45, 46, 47, 48, 49, 50, 51, 52, 53, 54, 55, 60, 70, 80, 90, 100, 150, 200, 250, 300, 350, 400, 411 or 482 variants selected from but not limited to those listed in FIG. 1.

In some embodiments, the reagents in the kit are included as lyophilized powders. In some embodiments, the reagents in the kit are included as lyophilized powders with instructions for reconstitution. In some embodiments, the reagents in the kit are included as liquids. In some embodiments, the reagents are included in plastic and/or glass vials or other appropriate containers. In some embodiments the primers and probes are all contained in individual containers in the kit. In some embodiments, the primers are packaged together in one container, and the probes are packaged together in another container. In some embodiments, the primers and probes are packaged together in a single container.

In some embodiments, the kit further includes control gDNA and/or DNA samples. In some embodiments, the control DNA sample is normal (e.g., from a subject who does not have KC). In some embodiments, the control DNA sample corresponds to the mutation being detected, including any of variants selected from the group listed in FIG. 1.

In some embodiments, the concentration of the control DNA sample is 5 ng/μL, 10 ng/μL, 20 ng/μL, 30 ng/μL, 40 ng/μL, 50 ng/μL, 60 ng/μL, 70 ng/μL, 80 ng/μL, 90 ng/μL, 100 ng/μL, 110 ng/μL, 120 ng/μL, 130 ng/μL, 140 ng/μL, 150 ng/μL, 160 ng/μL, 170 ng/μL, 180 ng/μL, 190 ng/μL or 200 ng/μL. In some embodiments, the concentration of the control DNA sample is 50 ng/μL, 100 ng/μL, 150 ng/μL or 200 ng/μL. In some embodiments, the concentration of the control DNA sample is 100 ng/μL. In some embodiments, the control DNA samples have the same concentration. In some embodiments, the control DNA samples have different concentrations.

In some embodiments, the kit can further include buffers, for example, GTXpress TAQMAN® reagent mixture, or any equivalent buffer. In some embodiments, the buffer includes any buffer described herein.

In some embodiments, the kit can further include reagents for use in cloning, such as vectors (including, e.g., M13 vector).

In some embodiments, the kit further includes reagents for use in purification of DNA.

In some embodiments, the kit further includes instructions for using the kit for the detection of corneal dystrophy in a subject. In some embodiments, these instructions include various aspects of the protocols described herein.

What is claimed:

1. A method for developing a treatment regimen for the treatment of keratoconous (KC) in a subject, the method comprising diagnosing or prognosing KC in the subject in accordance with detecting genetic variants of ten or more genes in a sample from the subject, wherein:
the ten or more genes are selected from the group consisting of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469; and
the presence of the genetic variants is indicative of the need for a KC treatment regimen in the subject.

2. The method according to claim 1, wherein said detecting comprises detecting at least one genetic variant from each of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469.

3. The method according to claim 1, wherein said variant detection is by a sequencing method.

4. The method according to claim 1, wherein the genetic variants are selected from the group consisting of genetic variants listed in FIG. 1.

5. The method according to claim 1, wherein the genetic variants are hundred or more variants selected from the group consisting of genetic variants listed in FIG. 1.

6. The method according to claim 1, wherein the genetic variants comprise 411 variants of FIG. 1.

7. The method according to claim 1, wherein the genetic variants comprise 482 variants including the genetic variants listed in FIG. 1.

8. The method according to claim 1, wherein the subject is African, Caucasian, Hispanic, or East Asian.

9. The method according to claim 1, further comprising amplifying a nucleotide molecule from the sample from the subject.

10. The method according to claim 1, wherein the detecting comprises detecting the genetic variants in a nucleotide molecule from the sample from the subject or its amplicons.

11. A method for treating keratoconous (KC) in a subject, the method comprising:
diagnosing or prognosing KC in a subject or predicting risk of KC in the subject in accordance with detecting genetic variants of five or more genes in a sample from the subject, wherein the five or more genes are selected from a first group consisting of ABCA4, ABCB5, ABCC6, ADAMTS18, ADGRV1, AGBL1, ANGPTL7, BEST1, CHST6, COL2A1, COL4A1, COL4A2, COL4A3, COL4A4, COL5A1, COL5A2, COL6A1, COL8A2, COL12A1, COL17A1, CYP4V2, DIAPH1, DOCK9, FOXE3, FYN, GJA8, GSN, HGF, ILIA, ILIRN, IL6, IL10, ITGB1, KERA, KRT3, KRT12, KRT13, KRT15, KRT16, KRT23, KRT24, LCAT, LOX, LRRN1, LTBP2, MAP2K1, MAP3K19, MTOR, MYLK, NLRP1, OVOL2, PAX6, PIK3CG, PIKFYVE, PIK3R1, PRDM5, PTK2, PXDN, PXN, RAF1, RHOA, SFTPD, SHC1, SIX5, SLC4A11, TACSTD2, TCF4, TGFBI, TLN1, UBIAD1, VSX1, WNT9A, WNT9B, ZEB1, and ZNF469, including detecting one or more variants of at least one gene selected from a second group consisting of ABCA4, ABCB5, ABCC6, ADAMTS18, ADGRV1, ANGPTL7, BEST1, COL2A1, COL4A1, COL4A2, COL4A3, COL4A4, COL5A2, COL6A1, COL12A1, DIAPH1, DOCK9, FYN, HGF, ILIA, ILIRN, IL6, IL10, ITGB1, KRT13, KRT15, KRT16, KRT23, KRT24, LOX, LRRN1, LTBP2, MAP2K1, MAP3K19, MTOR, MYLK, NLRP1, PAX6, PIK3CG, PIK3R1, PTK2, PXDN, PXN, RAF1, RHOA, SFTPD, SHC1, SIX5, TLN1, WNT9A, and WNT9B and wherein the presence of the genetic variants is indicative of a diagnosis or prognosis of KC in the subject; and
administering a treatment for KC to the subject.

12. The method according to claim 11, including detecting genetic variants of ten or more genes selected from the second group.

13. The method according to claim 11, wherein said variant detection is by a sequencing method.

14. The method according to claim 11, wherein the genetic variants are selected from the group consisting of genetic variants listed in FIG. 1.

15. The method according to claim 11, wherein the genetic variants are hundred or more variants selected from the group consisting of genetic variants listed in FIG. 1.

16. The method according to claim 11, wherein the genetic variants comprise 411 variants of FIG. 1.

17. The method according to claim 11, wherein the genetic variants comprise 482 variants including the genetic variants listed in FIG. 1.

18. The method according to claim 11, wherein the subject is African, Caucasian, Hispanic, or East Asian.

19. The method according to claim 11, further comprising amplifying a nucleotide molecule from the sample from the subject.

20. The method according to claim 11, wherein the detecting comprises detecting the genetic variants in a nucleotide molecule from the sample from the subject or its amplicons.
